SEQUENCE LISTING (19 sequences)

SEQ ID NO: 1 - protein, 8 aa, synthetic construct
LRTGNNFQ

SEQ ID NO: 2 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - protein, 25 aa, synthetic construct
YCLEYFPSQMLRTGNNFQFTYTFED

SEQ ID NO: 4 - protein, 420 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFED

SEQ ID NO: 5 - protein, 318 aa, synthetic construct
VPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - protein, 210 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGA

SEQ ID NO: 7 - protein, 14 aa, synthetic construct
AADGYLPDWLEDNL

SEQ ID NO: 8 - protein, 6 aa, synthetic construct
SEGIRE

SEQ ID NO: 9 - protein, 23 aa, synthetic construct
WWALKPGAPKPKANQQKQDDGRG

SEQ ID NO: 10 - protein, 15 aa, synthetic construct
LVLPGYKYLGPFNGL

SEQ ID NO: 11 - protein, 24 aa, synthetic construct
DKGEPVNAADAAALEHDKAYDQQL

SEQ ID NO: 12 - protein, 16 aa, synthetic construct
QAGDNPYLRYNHADAE

SEQ ID NO: 13 - protein, 20 aa, synthetic construct
FQERLQEDTSFGGNLGRAVF

SEQ ID NO: 14 - protein, 12 aa, synthetic construct
QAKKRVLEPLGL

SEQ ID NO: 15 - protein, 220 aa, unidentified
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGAD

SEQ ID NO: 16 - protein, 12 aa, synthetic construct
QAKKRVLEPLGL

SEQ ID NO: 17 - protein, 528 aa, synthetic construct
PMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 18 - protein, 5 aa, synthetic construct
NNLTS

SEQ ID NO: 19 - protein, 16 aa, synthetic construct
SQMLRTGNNFQFTYTF

(19) United States
(12) Patent Application Publication
(10) Pub. No.: US 2025/0164488 A1
(43) Pub. Date: May 22, 2025

(54) STRUCTURE AND FUNCTION CHARACTERIZATION OF ADENO-ASSOCIATED VIRUS (AAV) BY HDX-MS

(71) Applicant: Regeneron Pharmaceuticals, Inc., Tarrytown, NY (US)

(72) Inventors: Haibo Qiu, Millwood, NY (US); Xiang Ye, Elmsford, NY (US); Yunli Hu, Tarrytown, NY (US); Ning Li, New Canaan, CT (US)

(21) Appl. No.: 18/949,497

(22) Filed: Nov. 15, 2024

Related U.S. Application Data
(60) Provisional application No. 63/600,481, filed on Nov. 17, 2023.

(51) Int. Cl. G01N 33/569; G01N 33/68

(52) U.S. Cl. CPC G01N 33/56983; G01N 33/6848; G01N 2333/95; G01N 2458/15

(57) ABSTRACT

The present disclosure generally pertains to methods for characterizing the structure stability and conformational dynamics of an AAV particle using Hydrogen-Deuterium Exchange Mass Spectrometry (HDX-MS).

### CROSS REFERENCE TO RELATED APPLICATIONS

The present application claims priority to and the benefit of U.S. patent application No. 63/600,481, “STRUCTURE AND FUNCTION CHARACTERIZATION OF ADENO-ASSOCIATED VIRUS (AAV) BY HDX-MS,” which was filed on Nov. 17, 2023, the disclosure of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Nov. 12, 2024, is named 086939501202_Sequence listing and is 20 kilobytes in size.

### BACKGROUND

Adeno-associated virus (AAV) is the most widely used viral vector for in vivo gene therapy applications. AAVs have low immunogenicity and can enable long-term, stable gene expression. The use of AAVs for gene therapy has created the need for analytical methods to monitor and characterize these products. Understanding the structural stability, packaging and AAV interactions with other proteins, will help to evolve gene therapy products.

Significant work is required to structurally characterize a protein of interest. In order to characterize the overall capsid of AAVs, several approaches have been employed, such as differential scanning fluorimetry (DSF), differential scanning calorimetry (DSC), circular dichroism (CD) and dynamic light scattering (DLS). However, these biophysical methods can only determine the overall AAV capsid properties, such as thermal stability, size, etc. X-ray crystallography and cryogenic electron microscopy (cryoEM) have been used to provide high resolution biophysical characterization of the structure of AAVs. However, these methods are time consuming and yield only a static snapshot of the AAV structure. Mass spectrometry-based structural proteomics techniques, such as hydrogen-deuterium exchange (HDX), have been implemented for structural information, direct measurement of structural dynamics and stability of many protein systems. No structural dynamics information has been reported on AAVs using these biophysical characterization methods. Furthermore, there is no structure information of any kind (static or dynamic) that has been reported on the unique region of VP1 (VP1u) and VP1/VP2 either. In order to overcome these issues, methods of characterizing AAVs have been developed using HDX-MS.

It will be appreciated that a need exists for methods to characterize AAVs to understand their structural stability and dynamics.

### SUMMARY

The present disclosure provides methods of characterizing an AAV viral particle, comprising: incubating a sample comprising the AAV viral particle with a deuterium labeling buffer to form a labeled sample, equilibrating a gel filtration column with greater than 4 M to about 8 M Guanidinium chloride (GndHCl), contacting the labeled sample with the GndHCl on the equilibrated gel filtration column to form a buffer exchanged sample, contacting the buffer exchanged sample with a hydrolyzing agent to form a digested sample, detecting the digested sample with a liquid chromatography-mass spectrometer to determine a mass of the labeled peptides, and analyzing the mass of the labeled peptides to characterize the AAV viral particle.

The present disclosure provides methods of characterizing at least one AAV viral particle and viral vector interaction site in a sample, comprising: incubating a sample comprising (i) an AAV viral particle and a viral vector and a sample comprising (ii) an AAV viral particle with a deuterium labeling buffer to form labeled samples, equilibrating a gel filtration column with Guanidinium chloride (GndHCl, contacting the labeled samples with the GndHCl on the equilibrated gel filtration column to form buffer exchanged samples, contacting the buffer exchanged samples with a hydrolyzing agent to form digested samples, detecting the digested samples with a liquid chromatography-mass spectrometer to determine a mass of the labeled peptides, analyzing the mass of the labeled peptides, and analyzing hydrogen-deuterium exchange to characterize an AAV viral particle and viral vector interaction site.

These, and other, aspects of the present disclosure will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present disclosure.

### BRIEF DESCRIPTION OF THE DRAWINGS

FIG. 1A illustrates and exemplary AAV8 capsid structure and hydrogen-deuterium exchange mass spectrometry (HDX-MS) workflow of adeno-associated virus (AAV). Surface of an AAV8 capsid on the left. Two subunits are shown in dark brown and teal that are two-fold rotational symmetry. Two surface exposed amino acid residues are also colored (N590 purple blue and G268 green) for all sixty subunits. Those that are engaged in the same symmetry arrangement (three-fold and five-fold) of the brown and teal subunits are connected by lines to form two pentagons and two triangles. The five-fold symmetry axes of the two pentagons are shown by arrows. On the right are the five-fold (top) and the threefold (bottom) symmetry unit of the teal subunit isolated from the capsid.

FIG. 1B is an exemplary schematic overview of HDX-MS experiments performed on AAV. GdnHCl denotes guanidinium chloride.

FIG. 1C illustrates the hydrogen deuterium exchange for the amide backbone hydrogens, according to an exemplary embodiment.

FIG. 2 illustrates a workflow for the hydrogen-deuterium exchange mass spectrometry, according to an exemplary embodiment.

FIG. 3 shows the number of peptides formed after pepsin digestion, according to an exemplary embodiment.

FIG. 4 shows the number increased number of peptides identified after gel-filtration by spin column and pepsin digestion, according to an exemplary embodiment.

FIG. 5 shows the number of unique peptides identified across VP1 unique (VP1u), VP1 and 2 shared (VP1/2) and VP3 common (VP3), according to an exemplary embodiment.

FIG. 6 shows the comparison of the total ion chromatograms with and without the utilization of the gel-filtration spin column, according to an exemplary embodiment.

FIG. 7A shows the isotope patterns of individual peptides on mass spectrometry, according to an exemplary embodiment.

FIG. 7B shows the deuterium uptake over time, according to an exemplary embodiment.

FIG. 7C shows the peptides identified, the degree of deuterium exchange over time and the three-dimensional ribbon diagram of the location of the peptide of interest on VP1, according to an exemplary embodiment.

FIG. 8A shows a heat map for the incorporation of deuterium into AAV, according to an exemplary embodiment.

FIG. 8B shows a heat map for the incorporation of deuterium into AAV, according to an exemplary embodiment.

FIG. 8C shows a three-dimensional ribbon diagram of the AAV capsid protein VP3 colored according to the heat map data, according to an exemplary embodiment.

FIG. 9 shows a heat map for the VP1 unique region, according to an exemplary embodiment.

FIG. 10 shows the comparison of deuterium incorporation at different pH and temperatures, according to an exemplary embodiment.

FIG. 11A shows the deuterium uptake over time for the peptide composed of amino acids 2-15 from the unique region of VP1, according to an exemplary embodiment. * denotes a post-translational acetylation to the N-terminus amine group of this amino acid residue.

FIG. 11B shows the deuterium uptake over time for the peptide composed of amino acids 16-21 from the unique region of VP1, according to an exemplary embodiment.

FIG. 11C shows the deuterium uptake over time for the peptide composed of amino acids 22-44 from the unique region of VP1, according to an exemplary embodiment.

FIG. 11D shows the deuterium uptake over time for the peptide composed of amino acids 45-59 from the unique region of VP1, according to an exemplary embodiment.

FIG. 11E shows the deuterium uptake over time for the peptide composed of amino acids 60-83 from the unique region of VP1, according to an exemplary embodiment.

FIG. 11F shows the deuterium uptake over time for the peptide composed of amino acids 84-99 from the unique region of VP1, according to an exemplary embodiment.

FIG. 11G shows the deuterium uptake over time for the peptide composed of amino acids 100-119 from the unique region of VP1, according to an exemplary embodiment.

FIG. 11H shows the deuterium uptake over time for the peptide composed of amino acids 120-131 from the unique region of VP1, according to an exemplary embodiment.

FIG. 12 shows a predicted ribbon diagram of the unique region of VP1, according to an exemplary embodiment.

FIG. 13A, FIG. 13B, FIGS. 13C and 13D show the heat map of deuterium incorporation across the unique region of VP1 using differential HDX between the empty and full capsid (FIG. 13A) and the comparison of deuterium uptake between the empty and full capsid (FIG. 13B-D) across the unique region of VP1, according to an exemplary embodiment.

FIG. 14 shows the heat map of deuterium incorporation across VP1, VP2 and VP3 using differential HDX between the empty and full capsid, according to an exemplary embodiment.

FIG. 15 shows the level of change for the differential HDX heat map of VP3 mapped onto a surface represented structure model and the binding of a dAdC in the pocket, according to an exemplary embodiment.

FIG. 16 shows the ribbon diagram overlaid with the level of change of a VP3 subunit and the binding of a dAdC in the pocket, according to an exemplary embodiment.

FIG. 17 shows the electrostatic surface potential of VP3, according to an exemplary embodiment.

FIG. 18 shows the sequence of VP3 engaged in interacting with the DNA molecule as identified by cryoEM (top panel) and HDX (bottom panel), according to an exemplary embodiment.

FIG. 19 shows the surface represented model of the differential HDX heat map of VP3 and the potential surface of DNA interaction, according to an exemplary embodiment.

FIG. 20 shows the different HDX rates in a potential nucleotide binding site in VP3, according to an exemplary embodiment.

FIGS. 21A and 21B show AAV8 hydrogen-deuterium exchange (HDX) mapped onto the viral capsid structure. FIG. 21A shows a (Left panel) model of the AAV8 VP3 (amino acids 220-738) collected at 37° C. pH_{read} 7.5 with a 3.6 kb DNA payload. The percentage deuteron incorporation was deconvoluted from overlapping peptides and corrected for back exchange with respect to the control sample with maximal exchange. Back exchange corrected deuterium incorporation values were averaged over all time points and mapped. Middle and right panels, surface representation of a viral capsid with a single VP3 subunit. The middle shows the interior surface after removal of the rest of the capsid. The right shows an exterior view of exposed structural elements that exchange readily. FIG. 21B shows a deconvoluted heat map data as in FIG. 21A plotted against viral linear sequence, overlaid with the secondary structure and solvent accessible surface area. Secondary structures are shaded in green (α helices) and blue (β strands). The conserved secondary structural elements (α1 and βB-I) and nine variable regions (V-I to IX) are marked. Approximately the first 220 amino acids are not visible in any published structure, and thus no secondary structure information is overlaid with HDX data. The three types of symmetry axes (two-, three-, and five-fold) are marked by an eclipse, triangle, and pentagon, respectively. Solvent accessibility is measured by the relative accessible surface area (RSA) RSA was calculated for both a viral protein monomer by itself and as a part of a capsid. Regions show differences between curves (shaded in orange) at the inter-subunit interface. AAV, adeno-associated virus.

FIG. 22 is a panel of Relative deuterium uptake plots for nine representative peptides covering both VP1u and VP1/2. The uptake trajectories were obtained with two empty AAV8 samples and three full samples of AAV8 packed with the genome of interest with sizes of 3.4, 3.6, and 4.0 kb in size, as indicated on the plot. The labeling conditions are pH_{read} 6.6 and 10° C. In addition, plotted (black dashed line) is the theoretical intrinsic deuterium uptake curve with a protection factor of 10. Error bars represent 95% confidence interval of the data with replicates. AAV, adeno-associated virus.

FIGS. 23A-23B show VP1u populating as a heterogeneous conformation ensemble but interchanges concertedly across the entire region into a less protected and more unfolded state. FIG. 23A shows the isotopic distribution of a representative VP1u peptide. The labeling conditions are pH_{read} 6.6 and 10 C. °. Four conditions are shown: an empty capsid sample and three full capsid samples with DNA payloads of different sizes. For each condition, an hydrogen-deuterium exchange time series is shown, with label time increasing from top to bottom, from 10 s to 36 min. The last panel is the maximally deuterated control sample. The center of mass and population percentage for both populations are shown in each panel. The kinetics of interchange and degree of separation between the populations differ among the three full samples. The sample with 4 kb DNA payload has the largest separation and the slowest interchange rate between its more protected and less protected populations. FIG. 23B shows kinetic trajectories of interchange into the less protected state of different parts of VP1u in the presence of 4.0 kb DNA payload. The collection of peptides covers the entire VP1u. For each peptide, the fraction of the less protected population representing the more unfolded state is plotted against the labeling time. Error bars represents the range of the fitted population fractions from two independent time series replicates. The dotted black line shows if the interchange kinetic moves into the more unfolded state and follows a single exponential which is less stretched than the experimental kinetic trajectories.

FIGS. 24A-24C show the effect of DNA payload on capsid hydrogen-deuterium exchange. FIG. 24A is a heat map showing the difference in VP3 common region deuterium uptake between one of the empty capsid samples and one of the full capsid samples (3.6 kb DNA payload). The labeling conditions are pH_{read} 7.5 and 37° C. Numbers of deuterons are back exchange corrected and normalized to the number of exchangeable sites of the corresponding peptides. FIG. 24B The four regions showing substantial differences are marked on the viral protein structure model. The nucleotide binding sites are also shown. FIG. 24C shows relative deuterium uptake plots of selected peptides (one from each region). Error bars represent 95% confidence interval of the data with replicates.

FIGS. 25A-25C show potential DNA-viral protein interaction sites revealed by hydrogen-deuterium exchange (HDX). FIG. 25A shows differential HDX mapped onto the interior surface of a portion of a capsid consisting of subunits surrounding a five-fold axis and an adjacent three-fold axis. Mapped differential HDX was obtained by summation over all time points for a given peptide shown in FIG. 24A and averaged over all backbone amide sites within that peptide. The dinucleotides bound to the three-fold pocket are shown in sticks. A double headed arrow indicates a possible DNA binding trajectory that spans from the three-fold pocket and five-fold axis region. FIG. 25B shoes surface charge distribution mapped onto the same patch of a capsid. Even after discounting the false N-terminus at the five-fold channel, high electrostatic potential arises from several positively charged amino acids in the vicinity (K324, R407, etc.). FIG. 25C shows surface exposed amino acid residues in the four regions with substantial differential deuterium uptake, identified by a relative accessible surface area (RSA) greater than 0.3.

### DETAILED DESCRIPTION

The present disclosure provides a method of characterizing an AAV viral particle, comprising:
(a) incubating a sample comprising the AAV viral particle with a deuterium labeling buffer to form a labeled sample;
(b) equilibrating a gel filtration column with greater than 4 M to about 8 M Guanidinium chloride (GndHCl);
(c) contacting the labeled sample with the GndHCl on the equilibrated gel filtration column to form a buffer exchanged sample;
(d) contacting the buffer exchanged sample with a hydrolyzing agent to form a digested sample;
(e) detecting the digested sample with a liquid chromatography-mass spectrometer to determine a mass of the labeled peptides; and
(f) analyzing the mass of the labeled peptides to characterize the AAV viral particle.

The present disclosure provides a method of characterizing at least one AAV viral particle and viral vector interaction site in a sample, comprising:
(a) incubating a sample comprising (i) an AAV viral particle and a viral vector and a sample comprising (ii) an AAV viral particle with a deuterium labeling buffer to form labeled samples;
(b) equilibrating a gel filtration column with Guanidinium chloride (GndHCl);
(c) contacting the labeled samples with the GndHCl on the equilibrated gel filtration column to form buffer exchanged samples;
(d) contacting the buffer exchanged samples with a hydrolyzing agent to form digested samples;
(e) detecting the digested samples with a liquid chromatography-mass spectrometer to determine a mass of the labeled peptides;
(f) analyzing the mass of the labeled peptides; and
(g) analyzing hydrogen-deuterium exchange to characterize an AAV viral particle and viral vector interaction site.

In some embodiments, the gel filtration column is equilibrated with about 6 M to about 8 M Guanidinium chloride (GndHCl).

In some embodiments, the gel filtration column is equilibrated with phosphate.

In some embodiments, the gel filtration column is equilibrated with phosphate at a concentration of from about 25 mM to about 75 mM.

In some embodiments, the pH of the gel filtration column is about pH 2.0 to about pH 3.0.

In some embodiments, the sample is diluted with trifluoroacetic acid (TFA) to a final concentration below 2M GndHCl after steps (a)-(c).

In some embodiments, the hydrolyzing agent is pepsin.

In some embodiments, the hydrolyzing agent is immobilized over a resin column.

In some embodiments, two immobilized pepsin columns are connected in tandem.

In some embodiments, the liquid chromatography is coupled to the mass spectrometer.

In some embodiments, the liquid chromatography has an integrated cold box, maintaining temperature of about −5° C. to about 5° C.

In some embodiments, the liquid chromatography has an integrated cold box, maintaining temperature of about 0° C.

In some embodiments, contacting the sample with the GndHCl comprises decreasing the pH.

In some embodiments, contacting the sample with the GndHCl comprises decreasing the temperature.

In some embodiments, said liquid chromatography is reverse-phase high performance liquid chromatography.

In some embodiments, two chromatography columns are connected in tandem.

In some embodiments, the first column is a C8 column and/or the second column is a C18 column.

In some embodiments, said at least one AAV viral particle comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

In some embodiments, approximately 80% of the peptides analyzed exhibited less than or equal to 25% loss of deuterium labeling.

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub-combination.

Adeno-associated viruses (AAVs) have been widely used as gene delivery vectors to deliver genetic material, such as delivering nucleic acids for gene therapy. AAVs provide advantages over other gene therapy treatments, including non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1-Unique N-Terminal Domain and Its Role in Capsid Trafficking, Journal of Virology, May 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

AAV-based therapy has additional advantages, including being non-toxic, having cell type-specific infection, and offering different serotypes with varying cell transduction efficiencies. A disadvantage is that AAV production, purification, and characterization are more complex compared to, for example, antibody therapies. Fully packaged AAVs consist of an icosahedral capsid containing an about 4.8 kb single-stranded genome. An empty capsid has a molecular weight of about 3750 kDa, while a full capsid with an about 4.7 kb single-stranded genome has a molecular weight of about 5100 kDa. The purity of AAVs is defined by several product-related impurities, including empty capsids, capsids containing partial or incorrect genomes, and aggregated or degraded capsid, as well as residual HCPs.

The present application provides methods to structurally characterize AAV particles. In some exemplary embodiments, the optimization of a hydrogen deuterium exchange workflow was utilized for structural characterization. In another exemplary embodiment, the characterization of conformational changes of an AAV particle was examined in both empty and full capsids.

In order to characterize the overall capsid of AAVs, several approaches have been employed, such as differential scanning fluorimetry (DSF), differential scanning calorimetry (DSC), circular dichroism (CD) and dynamic light scattering (DLS). However, these biophysical methods can only determine the overall AAV capsid properties such as thermal stability, size, etc. X-ray crystallography and cryogenic electron microscopy (cryoEM) have been used to provide high resolution biophysical characterization of the structure of AAVs. However, these methods are time consuming and yield only a static snapshot of the AAV structure. Mass spectrometry-based proteomics techniques, such as hydrogen-deuterium exchange (HDX), have been implemented for structural information, direct measurement of structural dynamics and stability of many protein systems. No structural dynamics information has been reported on AAVs using these biophysical characterization methods. Furthermore, no structural information of any kind (static or dynamic) has been reported on the unique region of VP1 (VP1u) and VP1/VP2 either. In order to overcome these issues, methods of characterizing AAVs have been developed using HDX-MS.

The present application provides characterization methods for gene therapy products, and specifically AAV products, using hydrogen deuterium exchange. In some exemplary embodiments, the methods characterize conformational changes of an AAV viral particle, the unique region of VP1 (VP1u) and the conformational changes that occur when the capsid is empty versus full. This approach can be applied to other gene therapy products and can be used to understand structural dynamics of VP1/VP2/VP3, as well as improve process development and product stability, and engineer novel capsids.

This disclosure provides methods to satisfy the aforementioned demands by providing methods to characterize AAV particles to understand structural stability and dynamics to further improve transduction efficiency, DNA packaging, reducing empty capsids and improve long term storage. Exemplary embodiments disclosed herein satisfy the aforementioned demands and the long-felt needs.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively. Reference to a particular numerical value includes at least that particular value, unless the context clearly dictates otherwise.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present disclosure, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” refers to a mixture of molecules that comprises at least a viral particle, such as an AAV particle, or an empty viral capsid, that is subjected to manipulation in accordance with the methods of the disclosure, including, for example, separating, analyzing, extracting, concentrating, profiling and the like. The sample can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration. In some specific exemplary embodiments, the drug product can be selected from manufactured drug product in the clinic, shipping, storage, or handling.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr.

A “vector,” as used herein, refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant viral vector” refers to a recombinant polynucleotide vector including one or more heterologous sequences (e.g., nucleic acid sequence not of viral origin).

A “recombinant AAV vector (rAAV vector)” refers to a polynucleotide vector including one or more heterologous sequences (e.g., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (e.g. AAV Rep and Cap proteins).

A “viral particle” refers to a particle composed of at least one viral capsid protein and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roseolovirus, Lymphocryptovirus, Macavirus, Percavirus, and Rhadinovirus.

A “capsid” is the protein shell of a virus, which encloses the genetic material. Three viral capsid proteins, VP1, VP1 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

As used herein, the term “gene therapy” is a method of treatment of a genetic disease by modifying or manipulating a gene of interest. The key step in gene therapy is efficient delivery of the vector to the appropriate tissue or cells. Non-limiting examples of gene therapy products can include plasmid DNA, viral vectors, non-viral vectors, bacterial vectors, human gene editing technology, and patient-derived cellular gene therapy products.

In some exemplary embodiments, the sample can be prepared prior to LC-MS analysis. Preparation steps can include reduction, denaturation, alkylation, dilution and/or digestion.

As used herein, the term “hydrogen-deuterium exchange” refers to deuterium labeling a protein of interest. The backbone amide hydrogens can exchange with deuterium, when incubated with deuterium. The rate at which that exchange occurs is dependent on the structure of the protein in a certain environment. The use of mass spectrometry can be used to determine the protein structures through precisely measuring the mass increase due to the incorporated deuterium.

As used herein, the term “labeling buffer” refers to the buffer containing deuterium, utilized to label the amide backbone hydrogens. Non-limiting examples of a labeling buffer include HEPES, NaCl and TCEP or MES, NaCl and TCEP. In some exemplary embodiments, the concentration of HEPES in the labeling buffer can be at least between about 1 mM to about 40 mM. In a specific aspect, the concentration of HEPES in the labeling buffer can be about 1 mM HEPES, about 5 mM HEPES, about 10 mM HEPES, about 15 mM HEPES, about 20 mM HEPES, about 25 mM HEPES, about 30 mM HEPES, about 35 mM HEPES or about 40 mM HEPES including any and all values in between. In some exemplary embodiments, the concentration of NaCl in the labeling buffer can be about 100 mM NaCl to about 200 mM NaCl. In a specific aspect, the concentration of NaCl can be at least about 100 mM NaCl, about 110 mM NaCl, about 120 mM NaCl, about 130 mM NaCl, about 140 mM NaCl, about 150 mM NaCl, about 160 mM NaCl, about 170 mM NaCl, about 180 mM NaCl, about 190 mM NaCl and about 200 mM NaCl including any and all values in between. In some exemplary embodiments, the concentration of TCEP in the labeling buffer can be at least between about 0.5 mM TCEP to about 1.5 mM TCEP. In a specific aspect, the concentration of TCEP can be at least about 0.5 mM TCEP, about 0.6 mM TCEP, about 0.7 mM TCEP, about 0.8 mM TCEP, about 0.9 mM TCEP, about 1 mM TCEP, about 1.1 mM TCEP, about 1.2 mM TCEP, about 1.3 mM TCEP, about 1.4 mM TCEP and about 1.5 mM TCEP, including any and all values in between.

In a further specific aspect, the pH of the labeling buffer containing HEPES, NaCl and TCEP can be pH 6.5, about pH 7.0, about pH 7.5, about pH 8.0, and about pH 8.5, including any and all values in between. In a further specific aspect, the temperature at which the labeling reaction takes place is between about 30° C. and about 40° C., about 31° C., about 32° C., about 33° C., about 34° C., about 35° C., about 36° C., about 37° C., about 38° C., or about 39° C., including any and all values in between.

In some exemplary embodiments, the concentration of MES in the labeling buffer can be at least between about 5 mM MES to about 40 mM MES. In a specific aspect the concentration of MES can be at least about 5 mM MES, about 10 mM MES, about 15 mM MES, about 20 mM MES, about 25 mM MES, about 30 mM MES, about 35 mM MES and about 40 mM MES including any and all values in between. In some exemplary embodiments, the concentration of NaCl in the labeling buffer can be about 100 mM NaCl to about 200 mM NaCl. In a specific aspect, the concentration of NaCl can be at least about 100 mM NaCl, about 110 mM NaCl, about 120 mM NaCl, about 130 mM NaCl, about 140 mM NaCl, about 150 mM NaCl, about 160 mM NaCl, about 170 mM NaCl, about 180 mM NaCl, about 190 mM NaCl and about 200 mM NaCl including any and all values in between. In some exemplary embodiments, the concentration of TCEP in the labeling buffer can be at least between about 0.5 mM TCEP to about 1.5 mM TCEP. In a specific aspect, the concentration of TCEP can be at least about 0.5 mM TCEP, about 0.6 mM TCEP, about 0.7 mM TCEP, about 0.8 mM TCEP, about 0.9 mM TCEP, about 1 mM TCEP, about 1.1 mM TCEP, about 1.2 mM TCEP, about 1.3 mM TCEP, about 1.4 mM TCEP and about 1.5 mM TCEP including any and all values in between.

In a further specific aspect, the pH of the labeling buffer can be about 6.0, about pH 6.1, about pH 6.2, about pH 6.3, about pH 6.4, about pH 6.5, about pH 6.6, about pH 6.7,about pH 6.8, about pH 6.9, and about pH 7.0, including any and all values in between. In a further specific aspect, the temperature at which the labeling reaction takes place is between about 5° C. and about 15° C., about 6° C., about 7° C., about 8° C., about 9° C., about 10° C., about 11° C., about 12° C., about 13° C., or about 14° C., including any and all values in between.

In an exemplary embodiment, the gel filtration column is equilibrated with guanidinium chloride (GndHCl). In a specific aspect, the concentration of the GndHCl can be from greater than 4 M GndHCl to about 8 M GndHCl. In a specific aspect, the concentration of the GndHCl can be at least between and including about 6 M GndHCl and about 8 M GndHCl. In a specific aspect, the concentration of GndHCl can be greater than 4 M such as at least about 4.5 M, about 5.0 M, about 5.5 M, about 6.0 M GndHCl, about 6.5 M GndHCl, about 7 M GndHCl, about 7.5 M GndHCl or about 8.0 M GndHCl, including any and all values in between and including greater than 4 M to about 8 M. In a specific aspect, the concentration of the GndHCl is 7 M GndHCl.

Without wishing to be bound by theory, the rate of denaturation of a protein sample is logarithmically related to the concentration of GndHCl.

In an exemplary embodiment, the gel filtration column can be equilibrated with a high concentration of urea. In a specific aspect, the concentration of urea can be at least between and including 3 M urea and about 9 M urea.

In a further specific aspect, the gel filtration column is equilibrated with phosphate. In a specific aspect, the concentration of phosphate can be at least between about 25 mM phosphate and about 75 mM phosphate. In a specific aspect, the concentration of phosphate can be about 25 mM phosphate, about 30 mM phosphate, about 35 mM phosphate, about 40 mM phosphate, about 45 mM phosphate, about 50 mM phosphate, about 55 mM phosphate, about 60 mM phosphate, about 65 mM phosphate, about 70 mM phosphate or about 75 mM phosphate, including any and all values in between.

In a further specific aspect, the pH of the gel filtration column is between about pH 2.0 to about pH 3.0. In a specific aspect, the pH of the gel filtration column is about pH 2.0,about pH 2.1, about pH 2.2, about pH 2.3, about pH 2.4, about pH 2.5, about pH 2.6, about pH 2.7, about pH 2.8, about pH 2.9 or about pH 3.0, including any and all values in between.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like TCEP (see below), or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. The methods of present disclosure can include contacting a quenched sample with a hydrolyzing agent. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” or “hydrolyzing agent” refers to any of a large number of different agents that can perform digestion (enzymatically and non-enzymatically) of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)). One or a combination of hydrolyzing agents can cleave peptide bonds in a protein or polypeptide, in a sequence-specific manner, generating a predictable collection of shorter peptides. The ratio of hydrolyzing agent to protein and the time required for digestion can be appropriately selected to obtain optimal digestion of the protein. When the enzyme to substrate ratio (E/S) is unsuitably high, the correspondingly high digestion rate will not allow sufficient time for the peptides to be analyzed by mass spectrometer, and sequence coverage will be compromised. On the other hand, a low E/S ratio would need long digestion and thus long data acquisition time. The enzyme to substrate ratio can range from about 1:0.5 to about 1:200. As used herein, the term “digestion” refers to hydrolysis of one or more peptide bonds of a protein.

There are several approaches to carrying out digestion of a protein in a biological sample using an appropriate hydrolyzing agent, for example, enzymatic digestion or non-enzymatic digestion. Conventional methods use a digestive enzyme in conditions and concentrations sufficient to completely digest all protein in a sample prior to LC-MS analysis.

One of the widely accepted methods for digestion of proteins in a sample involves the use of proteases. Many proteases are available and each of them have their own characteristics in terms of specificity, efficiency, and optimum digestion conditions. Proteases refer to both endopeptidases and exopeptidases, as classified based on the ability of the protease to cleave at non-terminal or terminal amino acids within a peptide. Alternatively, proteases also refer to the six distinct classes—aspartic, glutamic, and metalloproteases, cysteine, serine, and threonine proteases, as classified based on the mechanism of catalysis. The terms “protease” and “peptidase” are used interchangeably to refer to enzymes which hydrolyze peptide bonds. A person skilled in the art can choose an appropriate hydrolyzing agent for forming peptides to be analyzed using HDX-MS.

The methods of present disclosure include contacting a digested sample to a liquid chromatography-mass spectrometer to determine a mass of deuterium-labeled peptides.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

The liquid chromatography step is generally carried out at about 0° C. The sub-zero degrees are applied with glycols administered into the buffer against freezing. Liquid chromatography can be carried out at temperatures above and below 0° C. In an exemplary embodiment, the temperature of the liquid chromatography can be about −5° C. to about 5° C., including any and all values in between.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Sciex), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptides (Q1), and hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Sciex), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; providing more accuracy and attomole-level limits of detection and quantification; enabling the confident confirmation of the peptide identity with spectral library matching; reducing assay development time since no target transitions need to be preselected; and ensuring UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, identifying the protein, sequencing amino acids of the protein fragments, determining protein sequencing, quantifying the protein, locating post-translational modifications, identifying post translational modifications, or comparability analysis, or combinations thereof.

In some exemplary aspects, the mass spectrometer can use nanoelectrospray or nanospray ionization. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com/proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic) or Sequest (fields.scripps.edu/sequest).

An alternative embodiment of the present disclosure includes methods of characterizing at least one viral vector in a sample, comprising incubating a sample comprising said viral vector with a deuterium labeling buffer to form a labeled sample, contacting the labeled sample with a quenching buffer to form a quenched sample, contacting the quenched sample with a gel filtration column to form a buffer exchanged sample, contacting the buffer exchanged sample with a hydrolyzing agent to form a digested sample, contacting the digested sample to a liquid chromatography-mass spectrometer to determine a mass of the labeled peptides, and analyzing the mass of the labeled peptides to characterize the viral vector.

It is understood that the present disclosure is not limited to any of the aforesaid protein(s), therapeutic protein(s), antibody(s), recombinant protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present disclosure will be more fully understood by reference to the following examples. They should not, however, be construed as limiting the scope of the disclosure.

### ENUMERATED EMBODIMENTS

The following examples are illustrative, but not limiting, of the compounds, compositions and methods described herein. Other suitable modifications and adaptations known to those skilled in the art are within the scope of the following embodiments.

### Enumerated Embodiments Section I

Embodiment I-1. A method of characterizing an AAV viral particle, comprising:
   (a) incubating a sample comprising the AAV viral particle with a deuterium labeling buffer to form a labeled sample;
   (b) contacting the labeled sample with a quenching buffer to form a quenched sample;
   (c) contacting the quenched sample with a gel filtration column to form a buffer exchanged sample;
   (d) contacting the buffer exchanged sample to a hydrolyzing agent to form a digested sample;
   (e) contacting the digested sample to a liquid chromatography-mass spectrometer to determine a mass of the labeled peptides; and
   (f) analyzing the mass of the labeled peptides to characterize the AAV viral particle.
Embodiment I-2. The method of embodiment 1, wherein the labeling buffer comprises one or more of 4-(2-hydroxyethyl)-1-piperazineethanesulfonic acid (HEPES), NaCl, tris(2-caroxyethyl)phosphine (TCEP).
Embodiment I-3. The method of any of the preceding embodiments, wherein the concentration of the labeling buffer is comprised of one or more of 5-40 mM HEPES, 100-200 mM NaCl, 0.5-1.5 mM TCEP.
Embodiment I-4. The method of any of the preceding embodiments, wherein the pH of the buffer is at a range of pH 6.5 to pH 8.5.
Embodiment I-5. The method of any of the preceding embodiments, wherein the pH of the buffer is at pH 7.5.
Embodiment I-6. The method of any of the preceding embodiments, wherein the labeling buffer comprises one or more of 2-(N-morpholino)ethanesulfonic acid (MES), NaCl, TCEP.
Embodiment I-7. The method of any of the preceding embodiments, wherein the labeling buffer comprises one or more of 5-40 mM 2-(N-morpholino) ethanesulfonic acid (MES), 100-200 mM NaCl, 0.5-1.5 mM TCEP.
Embodiment I-8. The method of any of the preceding embodiments, wherein the pH of the buffer is between pH 6 and 7.
Embodiment I-9. The method of any of the preceding embodiments, wherein the pH of the buffer is pH 6.6.
Embodiment I-10. The method of any of the preceding embodiments, wherein the labeling reaction is carried out at about 30° C. to about 40° C.
Embodiment I-11. The method of any of the preceding embodiments, wherein the labeling reaction is carried out at about 37° C.
Embodiment I-12. The method of any of the preceding embodiments, wherein the labeling reaction is carried out in a buffer comprised of HEPES, NaCl and TCEP.
Embodiment I-13. The method of any of the preceding embodiments, wherein the labeling reaction is carried out at about 5° C. to about 15° C.
Embodiment I-14. The method of any of the preceding embodiments, wherein the labeling reaction is carried out at about 10° C.
Embodiment I-15. The method of any of the preceding embodiments, wherein the labeling reaction is carried out in a buffer comprised of MES, NaCl and TCEP.
Embodiment I-16. The method of any of the preceding embodiments, wherein the gel filtration column is equilibrated with about 6 M to about 8 M Guanidinium chloride (GndHCl).
Embodiment I-17. The method of any of the preceding embodiments, wherein the gel filtration column is equilibrated with phosphate.
Embodiment I-18. The method of any of the preceding embodiments, wherein the concentration is about 25 mM to about 75 mM phosphate.
Embodiment I-19. The method of any of the preceding embodiments, wherein the pH of the gel filtration column is about pH 2.0 to about pH 3.0.
Embodiment I-20. The method of any of the preceding embodiments, wherein the pH of the gel filtration column is about pH 2.5.
Embodiment I-21. The method of any of the preceding embodiments, wherein the sample is diluted with trifluoroacetic acid (TFA) to a final concentration below 2 M GndHCl after labeling and desalting.
Embodiment I-22. The method of any of the preceding embodiments, wherein the sample is diluted about fourfold.
Embodiment I-23. The method of any of the preceding embodiments, wherein the labeling reaction is for a timed duration of about 10 seconds to about 120 hours.
Embodiment I-24. The method of any of the preceding embodiments, wherein the hydrolyzing agent is pepsin.
Embodiment I-25. The method of any of the preceding embodiments, wherein the hydrolyzing agent is immobilized over a resin.
Embodiment I-26. The method of any of the preceding embodiments, wherein two immobilized pepsin columns are connected in tandem.
Embodiment I-27. The method of any of the preceding embodiments, wherein the liquid chromatography is coupled to the mass spectrometer.
Embodiment I-28. The method of any of the preceding embodiments, wherein the liquid chromatography has an integrated cold box, maintaining temperature of about −5° C. to about 5° C.
Embodiment I-29. The method of any of the preceding embodiments, wherein the liquid chromatography has an integrated cold box, maintaining temperature of about 0° C.
Embodiment I-30. The method of any of the preceding embodiments, wherein quenching said sample comprises decreasing the pH.
Embodiment I-31. The method of any of the preceding embodiments, wherein quenching said sample comprises decreasing the temperature.
Embodiment I-32. The method of any of the preceding embodiments, wherein said liquid chromatography is reverse-phase high performance liquid chromatography.
Embodiment I-33. The method of any of the preceding embodiments, wherein two chromatography columns are connected in tandem.
Embodiment I-34. The method of any of the preceding embodiments, wherein the first column is a C8 column.
Embodiment I-35. The method of any of the preceding embodiments, wherein the second column is a C18 column.
Embodiment I-36. The method of any of the preceding embodiments, wherein said at least one AAV viral particle comprises a serotype selected from a group consisting of AAV1, AAV2, AAV3, AAV4, AAV5, AAV6, AAV7, AAV8, AAV9, AAV10, AAV11, AAV12, variations thereof and combinations thereof.
Embodiment I-37. A method of characterizing at least one viral vector in a sample, comprising:
   (a) incubating a sample comprising said viral vector with a deuterium labeling buffer to form a labeled sample;
   (b) contacting the labeled sample with a quenching buffer to form a quenched sample;
   (c) contacting the quenched sample with a gel filtration column to form a buffer exchanged sample;
   (d) contacting the buffer exchanged sample with a hydrolyzing agent to form a digested sample;
   (e) contacting the digested sample to a liquid chromatography-mass spectrometer to determine a mass of the labeled peptides; and
   (f) analyzing the mass of the labeled peptides to characterize the viral vector.
Embodiment I-38. The method of any of the preceding embodiments, wherein the labeling buffer is comprised one or more of HEPES, NaCl, TCEP.
Embodiment I-39. The method of any of the preceding embodiments, wherein the concentration of the labeling buffer is comprises one or more of 5-40 mM HEPES, 100-200 mM NaCl, 0.5-1.5 mM TCEP.
Embodiment I-40. The method of any of the preceding embodiments, wherein the pH of the buffer is at a range of pH 6.5 to pH 8.5.
Embodiment I-41. The method of any of the preceding embodiments, wherein the pH of the buffer is at pH 7.5.
Embodiment I-42. The method of any of the preceding embodiments, wherein the labeling buffer is comprised of one or more of MES, NaCl, TCEP.
Embodiment I-43. The method of any of the preceding embodiments, wherein the concentration of the labeling buffer comprises one or more of 5-40 mM MES, 100-200 mM NaCl, 0.5-1.5 mM TCEP.
Embodiment I-44. The method of any of the preceding embodiments, wherein the pH of the labeling buffer is between pH 6.0 and pH 7.0.
Embodiment I-45. The method of any of the preceding embodiments, wherein the pH of the buffer is at pH 6.6.
Embodiment I-46. The method of any of the preceding embodiments, wherein the labeling reaction is carried out at about 30° C. to about 40° C.
Embodiment I-47. The method of any of the preceding embodiments, wherein the labeling reaction is carried out at about 37° C.
Embodiment I-48. The method of any of the preceding embodiments, wherein the labeling reaction is carried out in a buffer comprised of HEPES, NaCl and TCEP.
Embodiment I-49. The method of any of the preceding embodiments, wherein the labeling reaction is carried out at about 5° C. to about 15° C.
Embodiment I-50. The method of any of the preceding embodiments, wherein the labeling reaction is carried out at about 0° C.
Embodiment I-51. The method of any of the preceding embodiments, wherein the labeling reaction is carried out in a buffer comprised of MES, NaCl and TCEP.
Embodiment I-52. The method of any of the preceding embodiments, wherein the gel filtration column is equilibrated with about 6 M to about 8 M GndHCl.
Embodiment I-53. The method of any of the preceding embodiments, wherein the gel filtration column is equilibrated with phosphate.
Embodiment I-54. The method of any of the preceding embodiments, wherein the concentration is about 25 mM to about 75 mM phosphate.
Embodiment I-55. The method of any of the preceding embodiments, wherein the pH of the gel filtration column is about pH 2.0 to about pH 3.0.
Embodiment I-56. The method of any of the preceding embodiments, wherein the pH of the gel filtration column is about pH 2.5.
Embodiment I-57. The method of any of the preceding embodiments, wherein the sample is diluted with trifluoroacetic acid (TFA) to a final concentration below 2 M GndHCl after labeling and desalting.
Embodiment I-58. The method of any of the preceding embodiments, wherein the sample is diluted about fourfold.
Embodiment I-59. The method of any of the preceding embodiments, wherein the labeling reaction is for a timed duration of about 10 seconds to about 120 hours.
Embodiment I-60. The method of any of the preceding embodiments, wherein the hydrolyzing agent is pepsin.
Embodiment I-61. The method of any of the preceding embodiments, wherein the hydrolyzing agent is immobilized over a resin.
Embodiment I-62. The method of any of the preceding embodiments, wherein two immobilized pepsin columns are connected in tandem.
Embodiment I-63. The method of any of the preceding embodiments, wherein the liquid chromatography is coupled to the mass spectrometer.
Embodiment I-64. The method of any of the preceding embodiments, wherein the liquid chromatography has an integrated cold box, maintaining temperature of about −5°° C. to about 5° C.
Embodiment I-65. The method of any of the preceding embodiments, wherein the liquid chromatography has an integrated cold box, maintaining temperature of about 0° C.
Embodiment I-66. The method of any of the preceding embodiments, wherein quenching said sample comprises decreasing the pH.
Embodiment I-67. The method of any of the preceding embodiments, wherein quenching said sample comprises decreasing the temperature.
Embodiment I-68. The method of any of the preceding embodiments, wherein said liquid chromatography is reverse-phase high performance liquid chromatography.
Embodiment I-69. The method of any of the preceding embodiments, wherein two chromatography columns are connected in tandem.
Embodiment I-70. The method of any of the preceding embodiments, wherein the first column is a C8 column.
Embodiment I-71. The method of any of the preceding embodiments, wherein the second column is a C18 column.
Embodiment I-72. The method of any of the preceding embodiments, wherein said sample contains an AAV viral particle.
Embodiment I-73. The method of any of the preceding embodiments, wherein said AAV particle comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

### Enumerated Embodiments Section II

Embodiment II-1. A method of characterizing an AAV viral particle, comprising:
   (a) incubating a sample comprising the AAV viral particle with a deuterium labeling buffer to form a labeled sample;
   (b) equilibrating a gel filtration column with greater than 4 M to about 8 M Guanidinium chloride (GndHCl);
   (c) contacting the labeled sample with the GndHCl on the equilibrated gel filtration column to form a buffer exchanged sample;
   (d) contacting the buffer exchanged sample with a hydrolyzing agent to form a digested sample;
   (e) detecting the digested sample with a liquid chromatography-mass spectrometer to determine a mass of the labeled peptides; and
   (f) analyzing the mass of the labeled peptides to characterize the AAV viral particle.
Embodiment II-2. A method of characterizing at least one AAV viral particle and viral vector interaction site in a sample, comprising:
   (a) incubating a sample comprising (i) an AAV viral particle and a viral vector and a sample comprising (ii) an AAV viral particle with a deuterium labeling buffer to form labeled samples;
   (b) equilibrating a gel filtration column with Guanidinium chloride (GndHCl);
   (c) contacting the labeled samples with the GndHCl on the equilibrated gel filtration column to form buffer exchanged samples;
   (d) contacting the buffer exchanged samples with a hydrolyzing agent to form digested samples;
   (e) detecting the digested samples with a liquid chromatography-mass spectrometer to determine a mass of the labeled peptides;
   (f) analyzing the mass of the labeled peptides; and
   (g) analyzing hydrogen-deuterium exchange to characterize an AAV viral particle and viral vector interaction site.
Embodiment II-3. The method of any of the preceding embodiments, wherein the gel filtration column is equilibrated with about 6 M to about 8 M Guanidinium chloride (GndHCl).
Embodiment II-4. The method of any of the preceding embodiments, wherein the gel filtration column is equilibrated with phosphate.
Embodiment II-5. The method of any of the preceding embodiments, wherein the gel filtration column is equilibrated with phosphate at a concentration of from about 25 mM to about 75 mM.
Embodiment II-6. The method of any of the preceding embodiments, wherein the pH of the gel filtration column is about pH 2.0 to about pH 3.0.
Embodiment II-7. The method of any of the preceding embodiments, wherein the sample is diluted with trifluoroacetic acid (TFA) to a final concentration below 2M GndHCl after steps (a)-(c).
Embodiment II-8. The method of any of the preceding embodiments, wherein the hydrolyzing agent is pepsin.
Embodiment II-9. The method of any of the preceding embodiments, wherein the hydrolyzing agent is immobilized over a resin column.
Embodiment II-10. The method of any of the preceding embodiments, wherein two immobilized pepsin columns are connected in tandem.
Embodiment II-11. The method of any of the preceding embodiments, wherein the liquid chromatography is coupled to the mass spectrometer.
Embodiment II-12. The method of any of the preceding embodiments, wherein the liquid chromatography has an integrated cold box, maintaining temperature of about −5° C. to about 5° C.
Embodiment II-13. The method of any of the preceding embodiments, wherein the liquid chromatography has an integrated cold box, maintaining temperature of about 0°° C.
Embodiment II-14. The method of any of the preceding embodiments, wherein contacting the sample with the GndHCl comprises decreasing the pH.
Embodiment II-15. The method of any of the preceding embodiments, wherein contacting the sample with the GndHCl comprises decreasing the temperature.
Embodiment II-16. The method of any of the preceding embodiments, wherein said liquid chromatography is reverse-phase high performance liquid chromatography.
   Embodiment II-17. The method of any of the preceding embodiments, wherein two chromatography columns are connected in tandem.
Embodiment II-18. The method of any of the preceding embodiments, wherein the first column is a C8 column and/or the second column is a C18 column.
Embodiment II-19. The method of any of the preceding embodiments, wherein said at least one AAV viral particle comprises a serotype selected from a group consisting of AAV1, AAV2,AAV3, AAV4, AAV5, AAV6, AAV7, AAV8, AAV9, AAV10, AAV11, AAV12, variations thereof and combinations thereof.

Embodiment II-20. The method of any of the preceding embodiments, wherein approximately 80% of the peptides analyzed exhibited less than or equal to 25% loss of deuterium labeling.

### EXAMPLES

### Materials and Methods

### Reagents and Chemicals

Adeno-associated viral serotype 8 samples were produced by Regeneron Pharmaceuticals, Inc. (Tarrytown, NY). Deuterium oxide (99.9% atom % D), 4-(2-hydroxyethyl)-1-piperazineethanesulfonic acid (HEPES), 2-(N-morpholino)ethanesulfonic acid (MES), guanidinium chloride, sodium chloride, and monobasic sodium phosphate were purchased from Sigma Aldrich (St. Louis, MO). Tris(2-carboxyethyl)phosphine hydrochloride (TCEP), Trifluoroacetic acid (TFA), and Zeba™ spin desalting column with 7k MWCO were purchased from Thermo Fisher Scientific (Waltham, MA). LC-MS grade solvents were purchased from Honeywell (Charlotte, NC).

### Collection of HDX Labeling Time Points

All AAV samples were concentrated using 10 kDa-MWCO Amicon concentrator to approximately 10¹⁴ viral capsid titer (˜1 mg/ml), then buffer exchanged into either 20 mM HEPES, 150 mM NaCl, 1 mM TCEP, pH 7.5 or 20 mM MES, 150 mM NaCl, 1 mM TCEP, pH 6.6 by using Zeba™ spin desalting column immediately prior to HDX. To initiate exchange reactions, AAV samples were diluted tenfold into D₂O buffer of the same buffer composition and incubated at either 37° C. (20 mM HEPES, 150 mM NaCl, 1 mM TCEP, pH 7.5) or 10° C. (20 mM MES, 150 mM NaCl, 1 mM TCEP, pH 6.6) for timed duration. Samples were quenched by mixing 40 μl of partially deuterated AAV with ice cold 6-8 μl of 50 mM phosphate buffer to drop the pH to 2.5. Quenched samples were immediately applied to a spin desalting column preequilibrated with 7M guanidinium chloride (GndHCl) buffered by 50 mM phosphate at pH 2.5. Quenched AAV samples were recovered in the flowthrough after centrifuging at 1500 g for 2 min and were flash frozen and stored at −80° C.

### Preparation of Maximally Deuterated AAV Samples and Assessment of Back Exchange Level

Maximally deuterated AAV samples (allD control) were prepared by buffer exchanging into 7M deuterated guanidinium chloride (GndHCl-d₆) in 90% D20 for 4 hours at 0° C. GndHCl-d₆ was prepared by dissolving guanidinium chloride in D₂O up to 8M and followed by solvent removal by lyophilization for three rounds. After incubation, the maximally deuterated samples were subject to the same treatment as the partially deuterated samples during collection of timed points, e.g. mixing with quench buffer, buffer exchanged into 7M guanidinium chloride solution, flash frozen, and stored at −80° C. The samples were used to both characterize the back exchange level of the workflow and the system and to adjust deuteration values of HDX experiments.

### HDX Sample Protease Digestion and LC-MS

Deuterated AAV samples were thawed and incubated on ice for 3 minutes before diluted fourfold with 0.027% TFA at pH 2.5 to reduce the final GndHCl concentration below 2M. Then sample time points were injected into a house-built integrated LC system consisting of two immobilized pepsin columns connected in tandem (Novabioassay and Trajan Scientific & Medical), a C8 trap column (the Nest Group), and an analytical C18 column (Waters). The temperature of the entire unit was maintained at 0° C. For each time point, ˜200 μl were injected into the system where digestion and desalting were performed as follows: a flow rate of 60 μl/min for the first 5 min and then 200 μl/min for another 3 min with a buffer of 0.1% FA pH adjusted by TFA to 2.25. After desalting on the C8 trap column, peptides were roughly separated on the C18 analytical column with a flow rate of 40 μl/min and a gradient of 5%-35% buffer B (0.1% formic acid in acetonitrile) over 25 min using a Thermo Ultimate 3000 LC system. Peptides were eluted into a Q Exactive Plus Mass Spectrometer with the following settings: resolution at 70,000, AGC target 10⁶, maximum injection time 50 ms, scan range 220-2000 m/z. Undeuterated AAV samples were used to build a peptide register list which was achieved by running three iterative MS2 runs with the following setting: data-dependent acquisition, loop count 5, resolution 17,500, AGC target 10⁵, maximum injection time 100 ms, isolation window 4.0 m/z, CE 27.

### Undeuterated Peptide Identification

Pepsin peptides in tandem MS runs were identified by using Byonic (Protein Metrics) and Proteome Discoverer (Thermo). For both, the sequence of AAV8 VP1 and porcine Pepsin were included in the search library. Sample digestion parameters were set to non-specific. Precursor mass tolerance and fragment mass tolerance were both set to 10 ppm. Oxidation and N-terminal acetylation are enabled as being dynamic/variable. Peptides with corrected false discovery rate<0.01 were imported into HDExaminer 3 (Trajan Scientific & Medical).

### Deuterated Peptide Search and Analysis

Deuterated peptides in the peptide pool were searched and the corresponding extracted ion current chromatogram were integrated by HDExaminer 3 at each exchange time point. For unimodal peptides, deuteration levels were determined by subtracting mass centroids of deuterated peptides from undeuterated peptides and corrected for back exchange by allD control sample. For bimodal peaks, deuterated peptide isotopic distributions were exported from HDExaminer 3 and analyzed separately by a python script to extract the centroids and the population fractions of subpopulations.

### Example 1. Optimization Using Hydrogen Deuterium Exchange

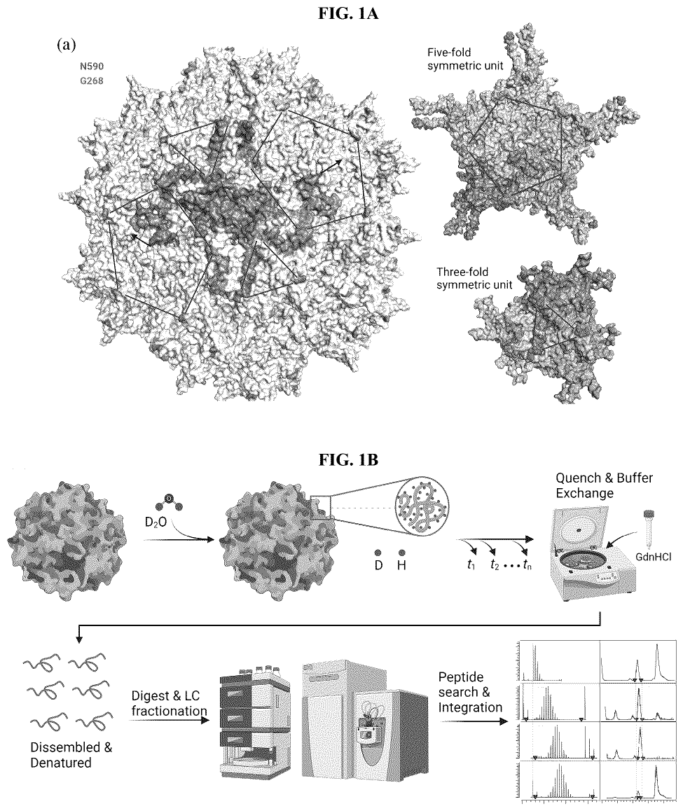

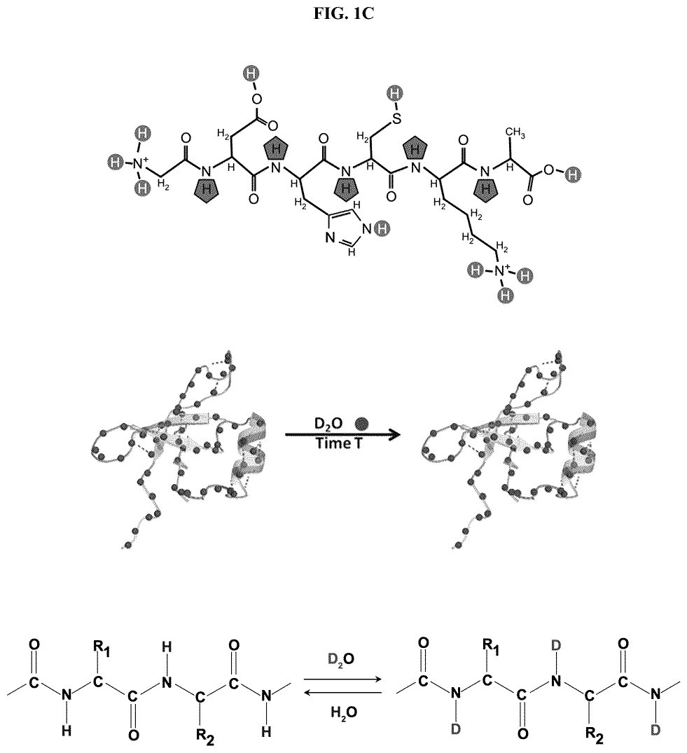

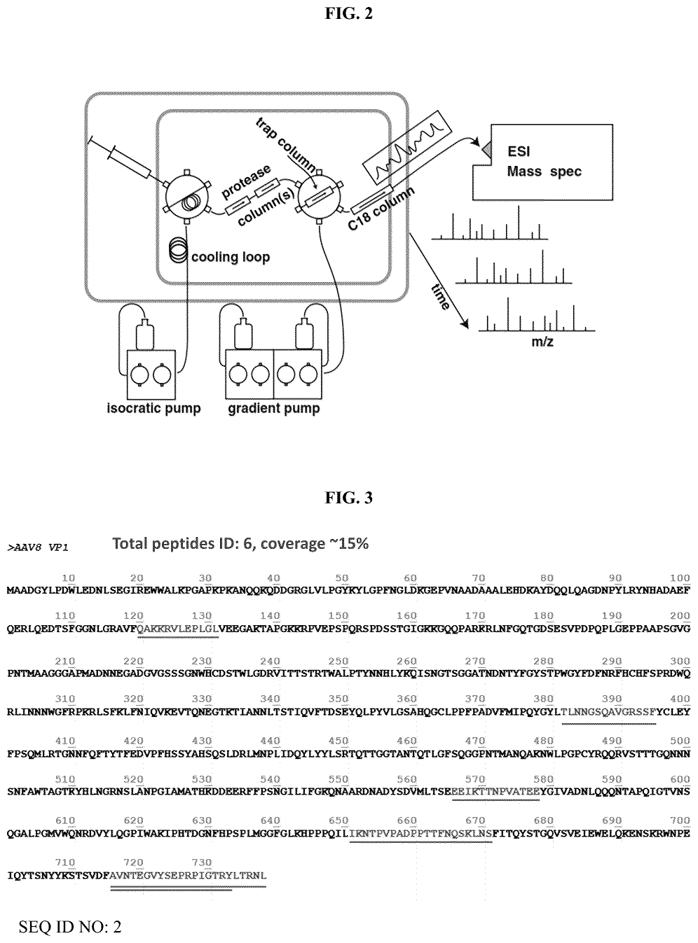

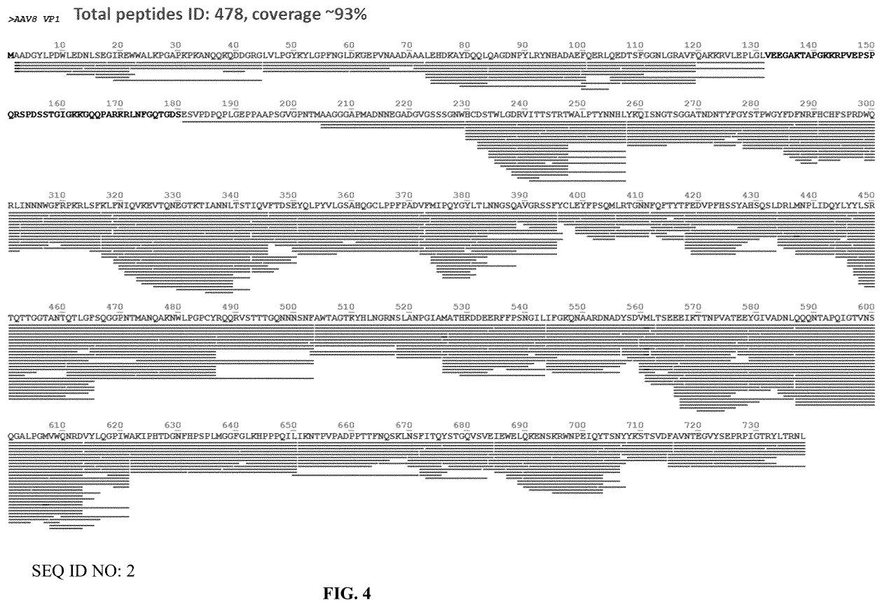

The characterization of AAVs in a sample presents a challenge due to, for example the capsids resistance to protease digestion, the inherent low abundance of VP1 and VP2 as compared to VP3 (a 1:1:10 ratio) and the low sample availability. The hydrogen-deuterium exchange mass spectrometry (HDX-MS) technique follows the time course of isotopic exchange between protein backbone amide hydrogen and solvent molecules (FIG. 1A-1B). The exchange rate of an amide site is affected primarily by the stability of its underlying structure and hence reports on the dynamics. An exemplary workflow of the labeling process is shown in FIG. 1C. The hydrogen atoms of the amide backbone are incubated with deuterium in solution. When exposed to deuterium, the hydrogen atoms are exchanged over a period of time. The labeled protein is then subjected to a quenching step through acidic pH and a decrease in temperature, prior to digestion on an immobilized protease column, desalting, fractionation by reverse-phase liquid chromatography (RP-HPLC) and mass spectrometry, as shown in FIG. 2. A typical HDX-MS workflow resulted in approximately 15% coverage, with six peptides identified in a single MS2 run, as shown in FIG. 3, providing insufficient sequence coverage of VP1 unique (VP1u) or VP1 and VP2 shared (VP1/VP2). This suggests resistance to protease digestion, which poses an obstacle in the strategy of the structural characterization of AAVs. Therefore, further development of the HDX-MS workflow was explored.

In order to improve the identification of peptides generated after digestion, methods were developed to buffer exchange the AAV sample using gel-filtration by spin column, in addition to an adjustment in the concentration of buffer used prior to digestion. The pre-digestion phase of the HDX-MS method was improved as described below.

The effectiveness of changing the molarity of the buffer was evaluated. The initial experiments were carried out at a buffer concentration of 4M GndHCl. Increasing the buffer concentration from 4M GndHCl to 7M GndHCl resulted in an increase in peptide identification. In addition, the effectiveness of adding a gel-filtration spin column step was evaluated. The desalting spin column was implemented to perform a buffer exchange to 7M GndHCl, after the quenching reaction was complete.

Finally, the addition of the gel-filtration spin column and the adjustment in the molarity of the buffer was evaluated. As shown in FIG. 4, a vast improvement in peptide coverage was observed with the implementation of the gel-filtration spin column and the change in molarity of the GndHCl. In total, 478 peptides were identified, resulting in approximately 93% coverage of the VP1/VP2/VP3 sequence.

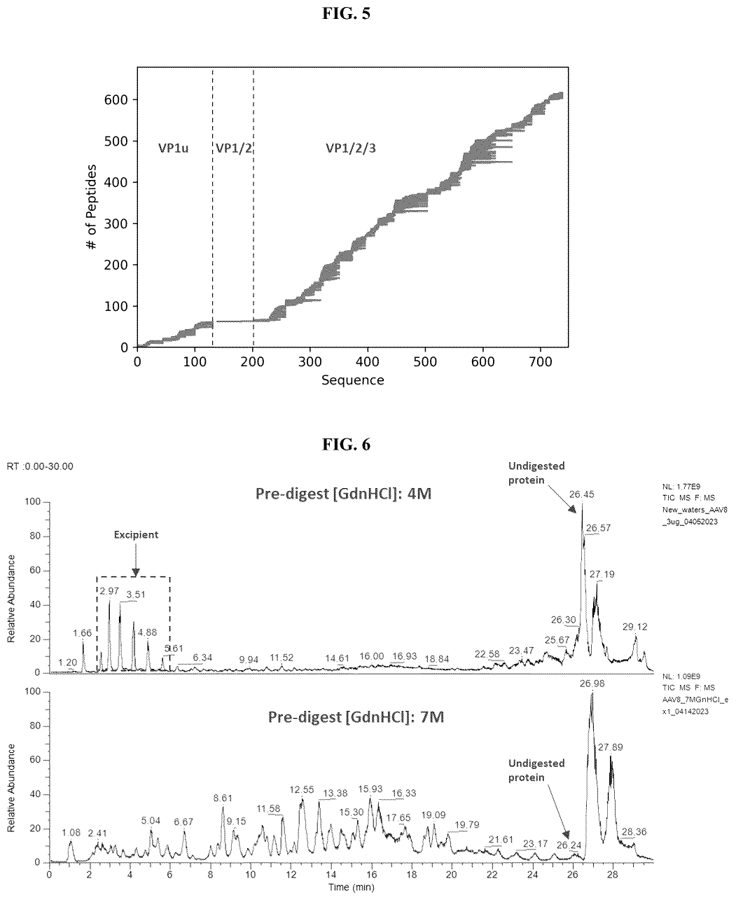

The optimized conditions were used to evaluate the reproducibility of the method of the present disclosure. Three iterative MS2 runs were performed, with 617 unique peptides being identified, for approximately 98% coverage of the VP1u, VP1/2 and VP3 common (VP3) sequence, as shown in FIG. 5. In addition to improved sequence coverage, approximately 80% of the peptides identified exhibited less than or equal to 25% loss of the deuterium label.

### Example 2. Characterization of AAV Using HDX-MS

The use of hydrogen deuterium exchange with mass spectrometry (HDX-MS) can be used to provide information on protein structure. Monitoring the levels of hydrogen deuterium exchange provides information about structure and conformation of proteins. For example, a low exchange rate observed can be interpreted as an inaccessible hydrogen atom on the amide backbone due to intramolecular bonding or protein structure.

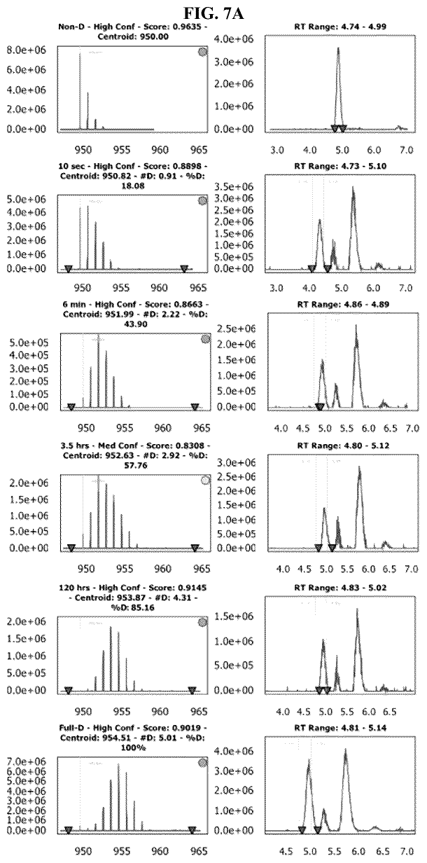

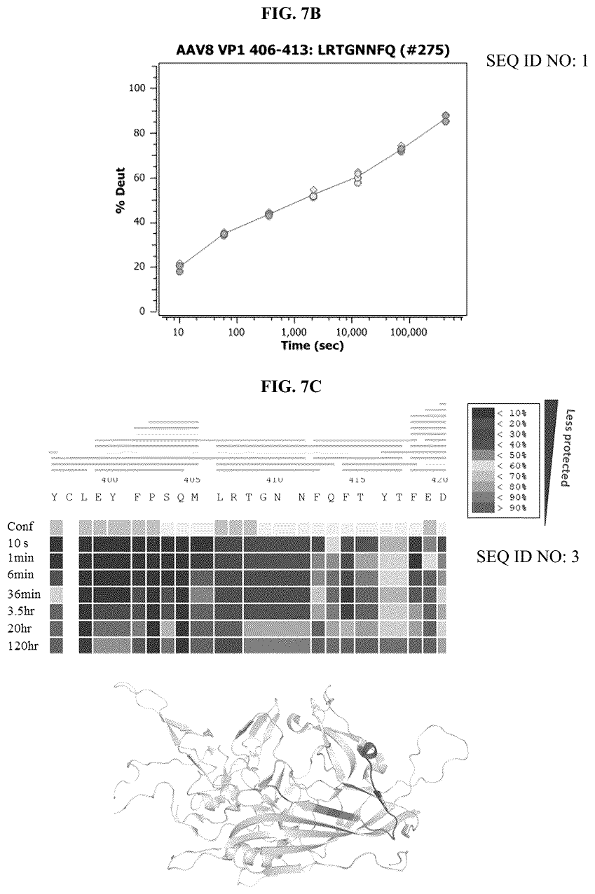

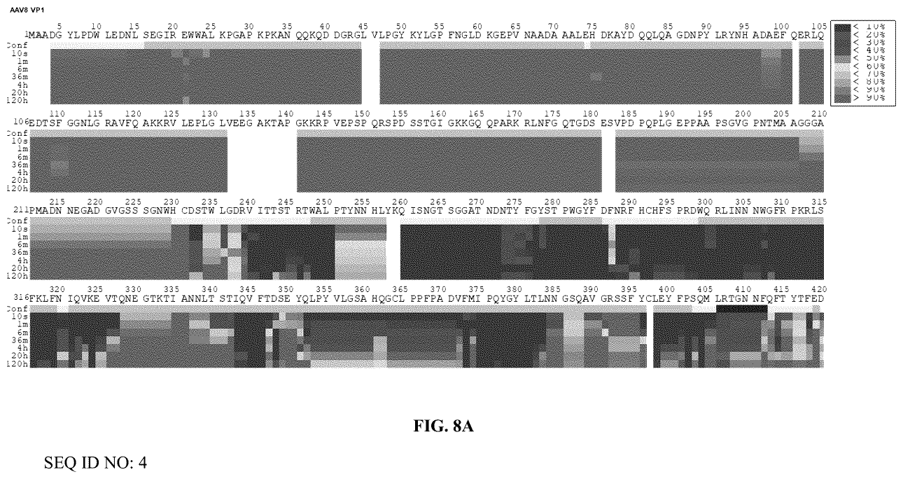

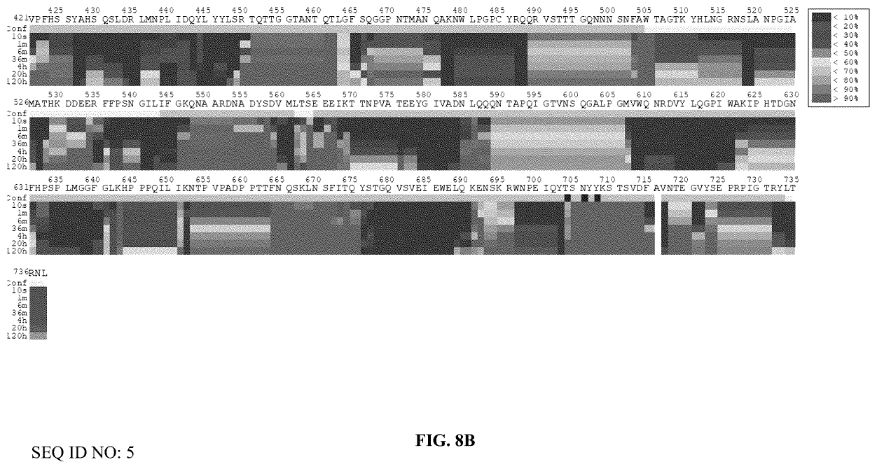

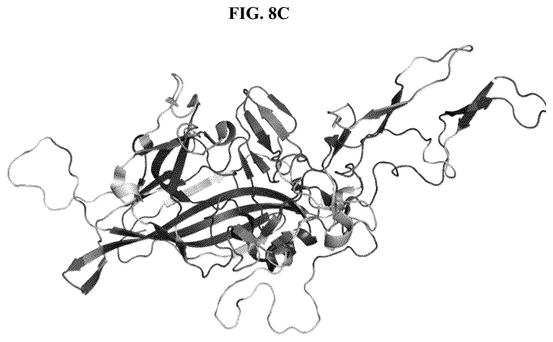

In one embodiment, the optimized conditions were used to structurally evaluate AAVs. Deuterium uptake and mass shifts were evaluated over time on peptide LRTGNNFQ (SEQ ID NO: 1), a sequence in VP1. FIG. 7A shows the isotope patterns of individual peptides with increasing mass over time, due to the increased uptake of deuterium. The peptide deuterium uptake plot reveals the hydrogen deuterium exchange of the individual peptide, as shown in FIG. 7B. The individual peptides segments generated are then overlaid and the degree of deuterium exchange is represented in a schematic showing the percent of hydrogen deuterium exchange based on level of protection, as shown in FIG. 7C. The most protected hydrogens represented on the heat map as blue, showing low exchange, and the least protected hydrogens represented on the heat map as red, showing high exchange. This data can then be mapped on a three-dimensional ribbon diagram of the protein to facilitate structural interpretation. The heat map for the hydrogen deuterium exchange incorporation for the AAV particle is shown in FIGS. 8A and 8B. The data was then mapped on a three-dimensional ribbon diagram of AAV capsid protein VP3 colored according to the heat map data, as shown in FIG. 8C.

### Example 3. Characterization of VP1u

Sequence analysis of VP1u indicates the presence of a phospholipase A2 (PLA2) like sequence. VP1u undergoes a conformational change during endocytosis, wherein it externalizes from inside the capsid to outside. It is theorized that PLA2 activity is believed to be essential for successful viral transduction.

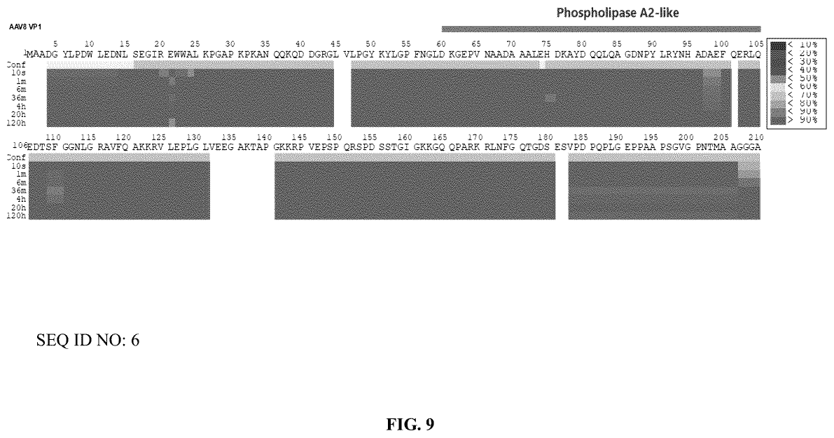

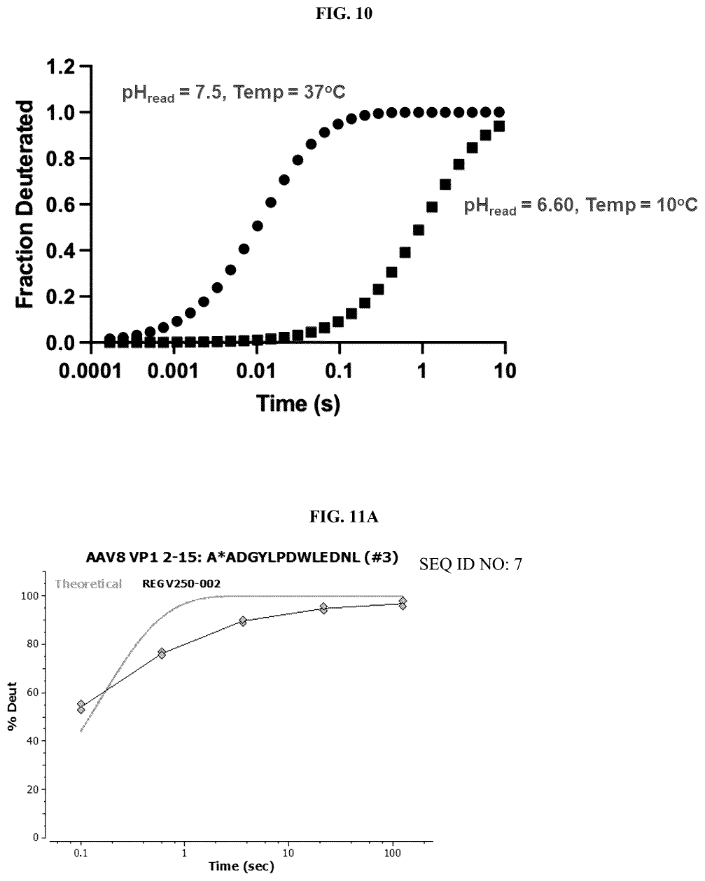

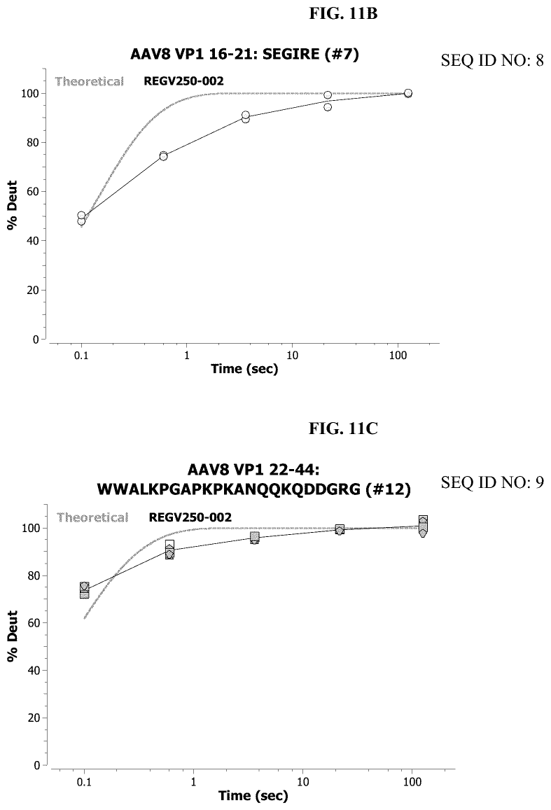

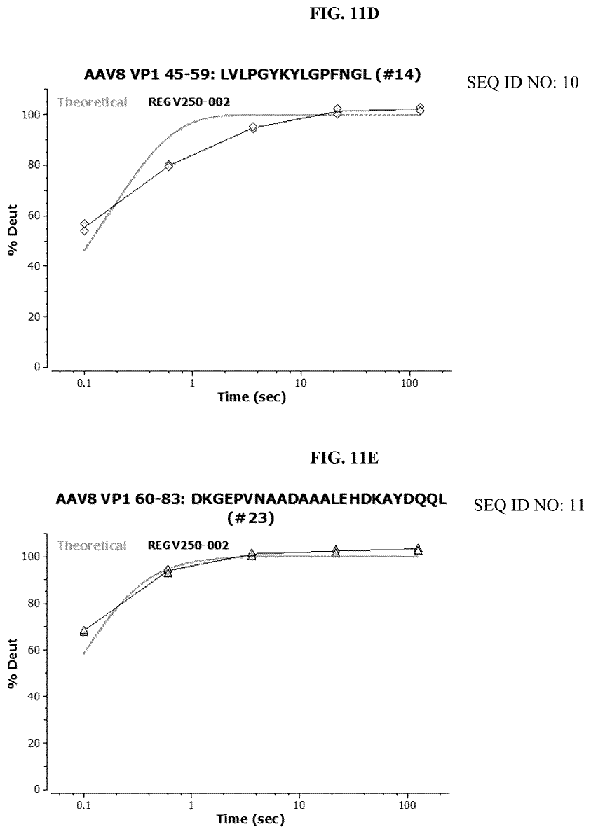

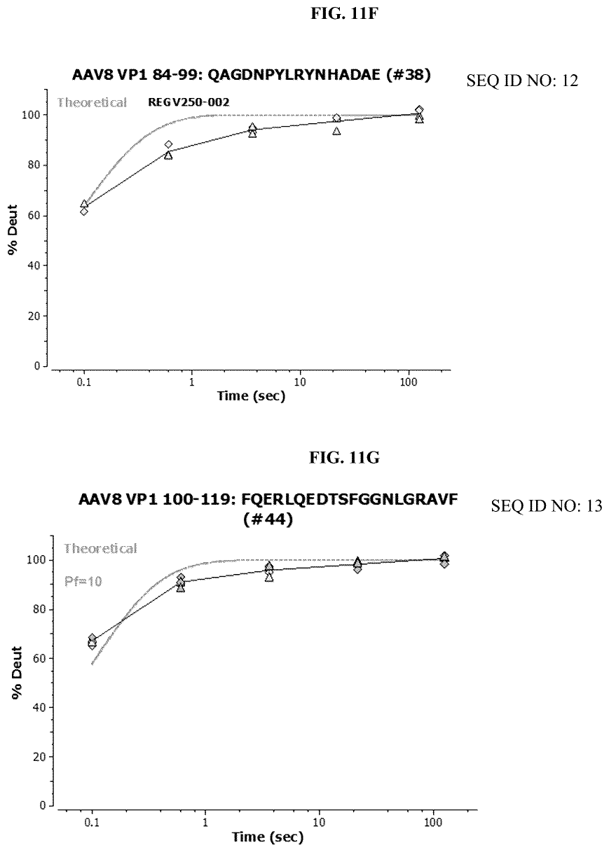

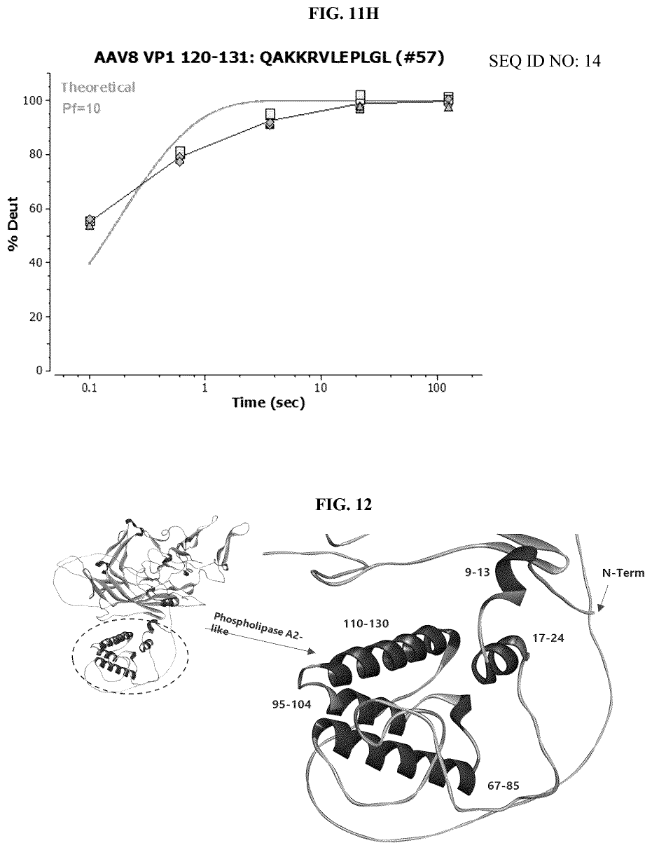

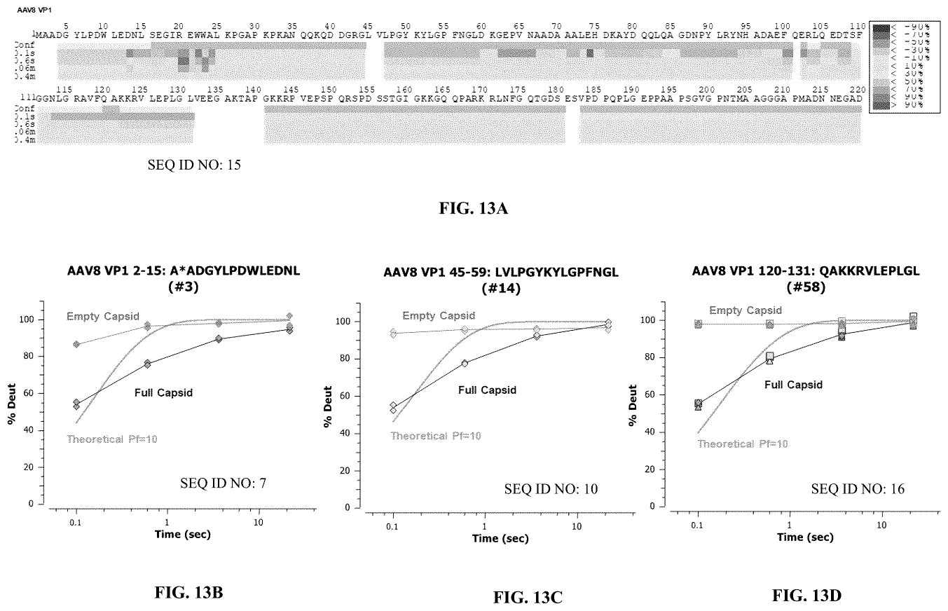

In one embodiment, the method of the present disclosure was used to structurally characterize VP1u. As shown in FIG. 9, there is a significant absence of protection in the VP1u region. By adjusting the pH and the temperature of the HDX reaction, the rate of exchange for the protein which gets deuterated can be decreased. As shown in FIG. 10, the simulated rate at which exchange occurs takes significantly longer at a lower pH and a lower temperature. This can be further observed in the peptide deuterium uptake maps for FIG. 11A-11H. The peptide plots show a fast exchange rate from hydrogen to deuterium, confirming the lack of protection for the entire VP1u region. The data suggests unstable nonspecific interaction and hydrogen bond formation in the PLA2 domain of the VP1u region. The corresponding heat map data was mapped on a predicted three-dimensional ribbon diagram of VP1, as shown in FIG. 12. The data suggests intermittent helical structure of the PLA2 domain.

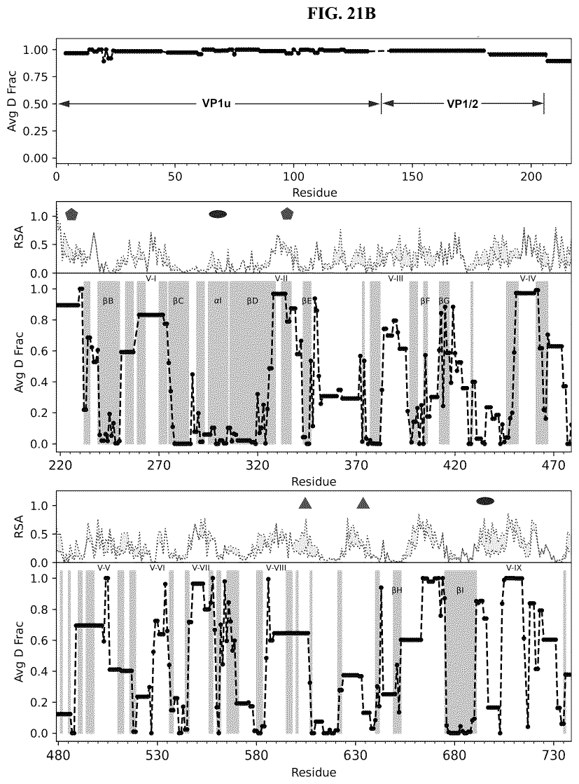

Deconvoluted HDX data calculated from overlapping peptides were mapped onto both the AAV8 three-dimensional structure as well as the linear amino acid sequence (FIG. 21). Amino acid sequence numbering of VP1, the longest viral protein (VP), was used and divided into three regions according to the overlapping patterns of the three viral proteins: (i) HDX information in VP1u was only contributed by VP1, (ii) HDX of VP1/2 sequence was contributed by both VP1 and VP2 proteins, and (iii) HDX of VP3 sequence came from all three viral proteins. Regions with stable secondary structures generally showed slower exchange than flexible loop regions (FIG. 21A). On the viral protein surface, areas exposed on the exterior of the capsid tended to be more flexible and to show faster exchange, whereas areas on the interior surface of the capsid were more rigid and showed slower exchange.

The structure of the ordered region of the VP monomer consists of a sequence-conserved eight-stranded antiparallel β-barrel motif (βB-βI) and an α-helix (αI), with long loop insertions between strands. Strands of the β-barrel motif, except one (βG), exchanged very slowly, some over the course of 120 h (FIG. 21B). Variable loop insertions (V-I to VIX) readily underwent HDX, and several showed complete exchange at the first or second time point (≤1 min).

At both the two- and three-fold symmetry axes, an extensive inter-subunit interface is present; consistently, structural elements in this area showed low structural dynamics and underwent HDX slowly (FIG. 21B). At the five-fold symmetry axis, a central channel is formed with a diameter of ˜20 Å at the widest point. Structural elements at the fivefold symmetry axis were more dynamic than those at the other two symmetry axes (FIG. 21B).

### Example 4. Characterization of Empty Versus Full Capsid

In one embodiment, the optimized method of the present disclosure was used to characterize the hydrogen deuterium exchange of empty versus full capsids to understand if conformational and/or structural changes occur. The peptide deuterium uptake plots for the PLA2 region of VP1u reveal the hydrogen deuterium exchange is slower in the full capsid than in the empty capsid, as shown in FIG. 13B-FIG. 13D. When the level of change identified in the differential HDX-MS between the empty capsid and full capsid was plotted on a heat map, significant change is observed when DNA is present inside the capsid compared to when it is absent. This data suggests DNA present in the capsid causes a conformational transition, for example spatial confinement/compression and a collapse of VP1u, leading to stabilization of VP1u. This data is consistent with examples documented in the literature, however, most of the literature is through theoretical simulation, and structural and conformation dynamics of VP1u had not been previously characterized. Further understanding this structural and conformational change of VP1u will enable a better understanding of the role of VP1 in AAV transduction.

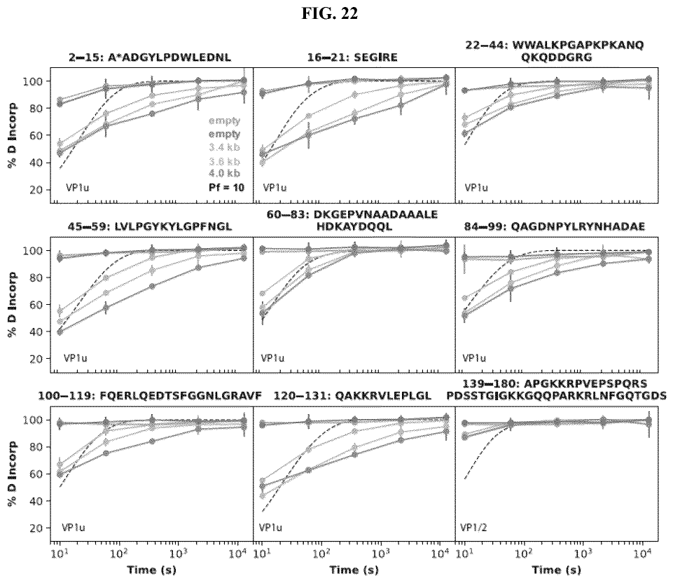

FIG. 22 shows centroid plots obtained under the labeling conditions (pH_{read} 6.6, 10_° C., <k_{chem}>˜1.2 s⁻¹) with a collection of high-quality peptides entirely covering VP1u and VP1/2. Five AAV8 samples were used: two empty and three filled with different DNA payloads of different sizes. VP1/2 showed no significant protection (Pf<3 for both EX2 and EX1 types of exchange) regardless of the size of the encapsulated DNA payload. In contrast, the HDX protection of VP1u varied depending on the presence and size of the DNA payload. For the two empty AAV8 samples, essentially no protection was detected (Pf˜1 for both EX2 and EX1 types of exchange). In contrast, all three full AAV8 samples showed some level of protection, which increased with increasing DNA payload size.

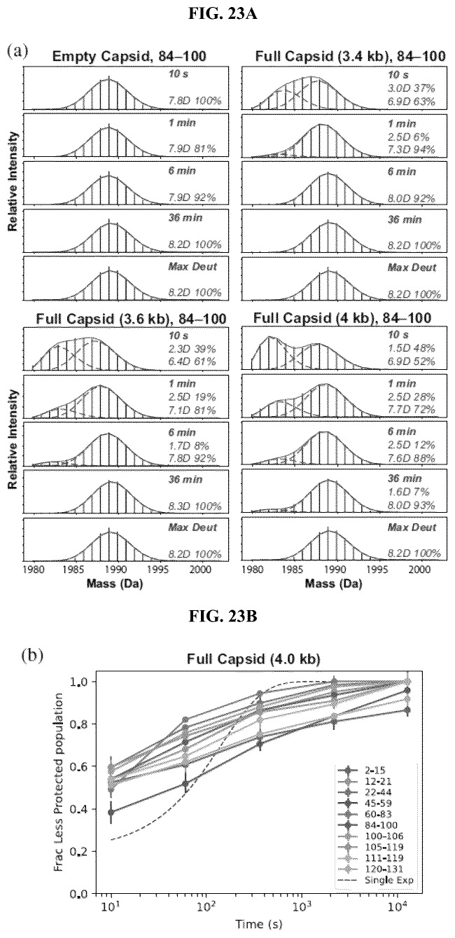

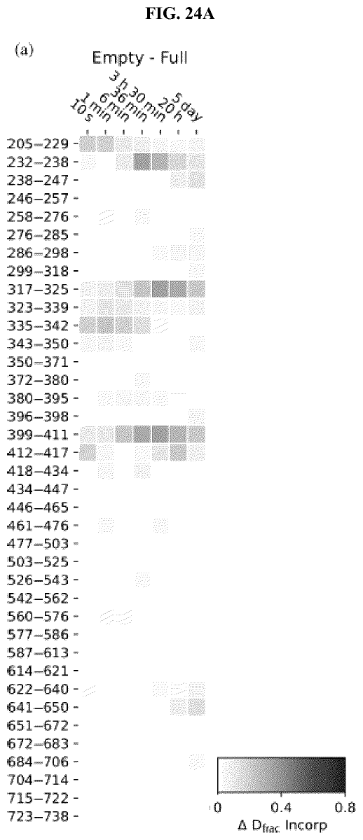

Different from other parts of the viral protein, the isotopic distributions of these deuterated peptides showed multimodal exchange (FIG. 23A) indicating populations of different stabilities. The separation between isotopic modes moderately improved as the size of DNA increased. FIG. 23B shows plots of the kinetic trajectories of conversion from the more protected population (lower mass) to the less protected population (higher mass) of the same set of VP1u peptides shown in FIG. 22.

For all peptides, the kinetic curves were more stretched than a single exponential (compare a simulated single exponential curve with the actual kinetic curves in FIG. 23B), indicating the existence of more than one kinetic event. In turn, it suggests that the conformation of the protected states was probably heterogeneous among different VP1s of the same capsid or VP1s of different capsids. Greater HDX protection was observed in the presence of a larger DNA payload, thereby causing stronger compression of the VP1u conformation (FIG. 23).

### Example 5. HDX-MS of VP-DNA Interaction

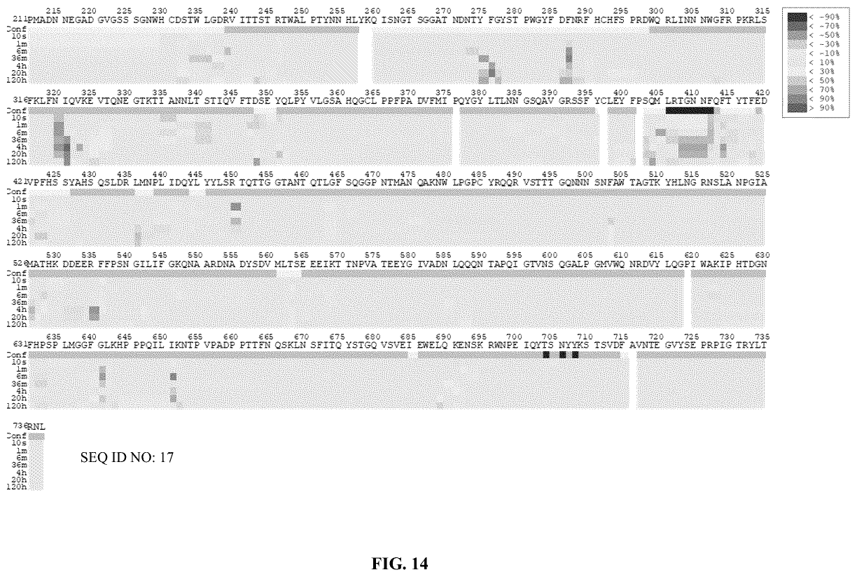

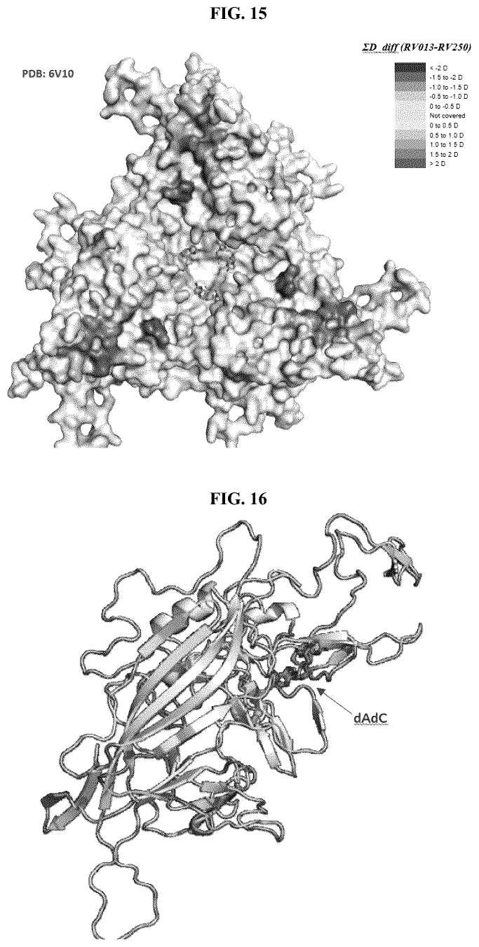

As described in Example 4, a conformational and structural change occurs to VP1u when a capsid contains DNA. In one embodiment, the optimized method was used to further characterize the VP-DNA interaction as the stability of the VP may be altered when DNA interacts with the VP. FIG. 14 shows the level of change identified in the differential HDX-MS between the empty capsid and full capsid plotted on a heat map for the VP1/VP2/VP3 sequence. Based on the differential HDX heat map, potential nucleic interaction sites were identified and mapped to a region in VP3. As shown in FIG. 15, the level of change identified in the differential HDX-MS between the empty capsid and full capsid was mapped to a three-dimensional structure model of multiple interacting VP3 subunits. Significant areas of conformational change are shown in red. FIG. 16 shows a single VP3 subunit mapped, as a ribbon diagram, with the level of change observed upon binding DNA.

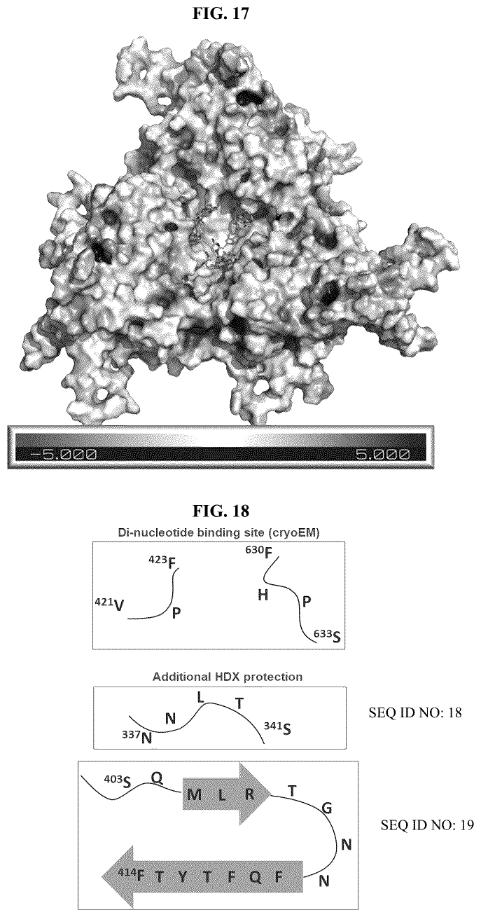

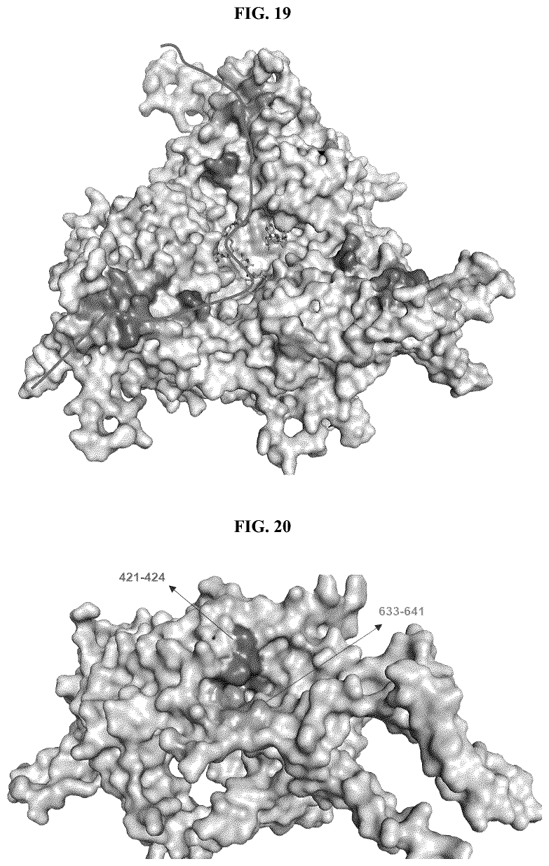

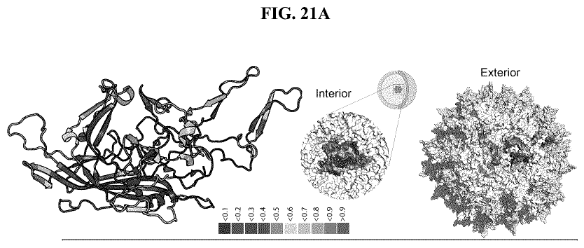

The surface represented model of the VP3 subunit mapped with the level of change for the differential heat map and binding sites for DNA is shown in FIG. 15. FIG. 17 shows the electrostatic surface potential of VP3, running from −5.000 (red) to +5.000 (blue). This representation is useful to help further characterize the DNA binding sites within AAV. The dinucleotide binding site, as identified by CryoEM, is shown in the top panel of FIG. 18. The potential additional DNA binding sites identified by HDX-MS are shown in the lower panel of FIG. 18. Based on the differential HDX heat map, the potential surface of DNA interaction is shown in FIG. 19. Further characterization of the putative binding site was carried out. Different HDX rates were identified in a potential nucleotide binding site, as shown in the surface represented model of FIG. 20.

This disclosure sets forth novel methods for the structural characterization of AAV using HDX-MS. There is limited information about AAV structure, stability and dynamics despite the availability of x-ray crystallography and cryoEM structures. Using HDX-MS, the methods of the present disclosure allowed for the resolve of the structural dynamics of AAV viral proteins, including significant conformational changes in VP1u. It was demonstrated that VP1u adopts an unfolded, collapsed state as a result of spatial confinement from DNA packed inside the capsid. Additionally, VP-DNA interaction sites previously resistant to characterization by high-resolution structural techniques were identified. Further understanding the structural and conformational changes that occur in AAVs can help to improve transduction efficiency by engineering novel viral vectors, improve DNA packaging efficiency to prevent empty capsid formation, and to improve the long-term storage of AAVs by increasing AAV stability.

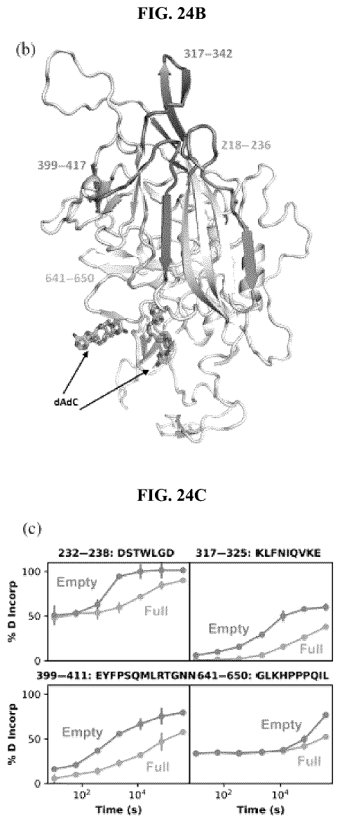

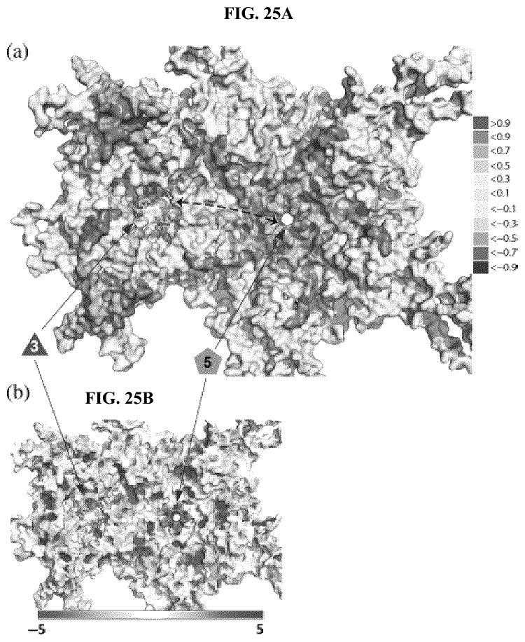

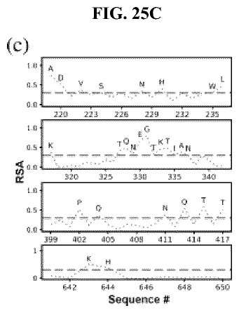

HDX-MS data of AAV8 samples with a 3.6 kb DNA payload or without it identified four regions showing substantial differences in deuteron uptake kinetics between the empty and full samples (FIG. 24). Two regions were DNA binding sites at the three-fold symmetry pocket, at amino acids 399-417 (β-strands F and G, and the intervening loop) and 641-650. Two regions also showed large differences, and both were at or near the five-fold symmetry axis, at amino acids 218-236 and 317-342 (β-strands D and E, and the intervening loop). The structurally unresolved N-terminus of the VP3 (amino acid 205-217) also shows large differential HDX. Mapping these segments onto the interior surface of the capsid showed that the five-fold channel and its vicinity on the interior surface are a hotspot for potential DNA-protein interaction (FIG. 25A). The surfaces of differential HDX regions radiate out from the fivefold axis and extend toward the three-fold pocket (marked by the double-headed arrow in FIG. 25A). The magnitude of HDX difference between the empty and full AAVs is similar among different payload sizes, indicating that the nature of such interaction is different from that which involves VP1u.

The nature of the interaction with DNA was examined at these sites by inspecting the surface charge distribution and types of surface exposed amino acids. Comparing differential HDX patterns with surface charge distribution revealed that the interaction at the five-fold axis, was at least partially attributable to favorable electrostatic interactions between the negatively charged phosphate backbone of DNA and the positively charged protein surface (FIG. 25B).

Beyond electrostatic potential, analysis of the surface exposed amino acid residues at these differential HDX segments suggested that polar-polar interactions may also contribute to DNA binding (FIG. 25C).

Instead of raising temperature or increasing time for denaturation which leads to high back-exchange, the methods developed herein involve a rapid buffer exchange step using a spin column to adjust the GdnHCl concentration to near its solubility limit (7M at 0° C.). At this high denaturant concentration, only a short incubation on ice is sufficient to destabilize the viral capsid proteins and enable efficient proteolytic digestion and MS measurement. The interior side of the five-fold channel and its surrounding regions (amino acids 218-223) are the prominent interaction sites and also have a net positive charge (FIGS. 25A and 25B). This interaction is driven by favorable electrostatic forces between the positively charged capsid surface and the negatively charged DNA phosphate backbone, and thus might not require a specific sequence of nucleotides.

What is claimed is:

1. A method of characterizing an AAV viral particle, comprising:
(a) incubating a sample comprising the AAV viral particle with a deuterium labeling buffer to form a labeled sample;
(b) equilibrating a gel filtration column with greater than 4 M to about 8 M Guanidinium chloride (GndHCl);
(c) contacting the labeled sample with the GndHCl on the equilibrated gel filtration column to form a buffer exchanged sample;
(d) contacting the buffer exchanged sample with a hydrolyzing agent to form a digested sample;
(e) detecting the digested sample with a liquid chromatography-mass spectrometer to determine a mass of the labeled peptides; and
(f) analyzing the mass of the labeled peptides to characterize the AAV viral particle.

2. The method of claim 1, wherein the gel filtration column is equilibrated with about 6 M to about 8 M Guanidinium chloride (GndHCl).

3. The method of claim 1, wherein the gel filtration column is equilibrated with phosphate.

4. The method of claims 1, wherein the gel filtration column is equilibrated with phosphate at a concentration of from about 25 mM to about 75 mM.

5. The method of claim 1, wherein the pH of the gel filtration column is about pH 2.0 to about pH 3.0.

6. The method of claim 1, wherein the sample is diluted with trifluoroacetic acid (TFA) to a final concentration below 2M GndHCl after steps (a)-(c).

7. The method of claim 1, wherein the hydrolyzing agent is pepsin.

8. The method of claim 1, wherein the hydrolyzing agent is immobilized over a resin column.

9. The method of claim 8, wherein two immobilized pepsin columns are connected in tandem.

10. The method of claim 1, wherein the liquid chromatography is coupled to the mass spectrometer.

11. The method of claim 1, wherein the liquid chromatography has an integrated cold box, maintaining temperature of about −5° C. to about 5° C.

12. The method of claim 1, wherein the liquid chromatography has an integrated cold box, maintaining temperature of about 0° C.

13. The method of claim 1, wherein contacting the sample with the GndHCl comprises decreasing the pH.

14. The method of claim 1, wherein contacting the sample with the GndHCl comprises decreasing the temperature.

15. The method of claim 1, wherein said liquid chromatography is reverse-phase high performance liquid chromatography.

16. The method of claim 15, wherein two chromatography columns are connected in tandem.

17. The method of claim 16, wherein the first column is a C8 column and/or the second column is a C18 column.

18. The method of claim 1, wherein said at least one AAV viral particle comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

19. The method of claim 1, wherein approximately 80% of the peptides analyzed exhibited less than or equal to 25% loss of deuterium labeling.

20. A method of characterizing at least one AAV viral particle and viral vector interaction site in a sample, comprising:
(a) incubating a sample comprising (i) an AAV viral particle and a viral vector and a sample comprising (ii) an AAV viral particle with a deuterium labeling buffer to form labeled samples;
(b) equilibrating a gel filtration column with Guanidinium chloride (GndHCl);
(c) contacting the labeled samples with the GndHCl on the equilibrated gel filtration column to form buffer exchanged samples;
(d) contacting the buffer exchanged samples with a hydrolyzing agent to form digested samples;
(e) detecting the digested samples with a liquid chromatography-mass spectrometer to determine a mass of the labeled peptides;
(f) analyzing the mass of the labeled peptides; and
(g) analyzing hydrogen-deuterium exchange to characterize an AAV viral particle and viral vector interaction site.
